(19) United States
(12) Patent Application Publication
(10) Pub. No.: US 2025/0032260 A1
(43) Pub. Date: Jan. 30, 2025

(54) CARDIAC DEVICE TESTING

(71) Applicant: VIVITRO LABS INC., Victoria (CA)

(72) Inventors: Yunquan CHEN, Delta (CA); Joseph Wesley MCMAHEN, Victoria (CA); Robert William FRASER, Victoria (CA); Luke Douglas BELL, Victoria (CA); Blake Oliver CROUCH, Esquimalt (CA); Willem Markus SCHOKKING, Victoria (CA); Tavia WALSH, Victoria (CA); Adriel Nissi NGO, Victoria (CA); Su Thida HTUN, Victoria (CA); Connor Richard BEWS, Surrey (CA); Benjamin Michael James TOMKA, Waterloo (CA)

(21) Appl. No.: 18/715,670

(22) PCT Filed: Dec. 1, 2022
(86) PCT No.: PCT/CA2022/051762

§ 371 Date: May 31, 2024

Related U.S. Application Data
(60) Provisional application No. 63/285,970, filed on Dec. 3, 2021.
(60) Provisional application No. 63/375,526, filed on Sep. 13, 2022.

(51) Int. Cl. A61F 2/24

(52) U.S. Cl. CPC A61F 2/2472

(57) ABSTRACT

An apparatus for testing cardiac devices comprises an inflow and an outflow chamber, a mount for supporting a prosthetic valve, and a fluid driving device operable to vary a pressure differential of a fluid between the inflow chamber and the outflow chamber in repeating cycles. The apparatus can comprise a bypass valve to modulate pressure characteristics across the prosthetic valve, reservoirs to maintain a pressure inside the apparatus, and compliance elements to modulate pressure characteristics in the apparatus. Methods for operating the apparatus include controlling the bypass valve to control testing characteristics across the prosthetic device, and methods for monitoring the pressure inside of the apparatus.

### REFERENCE TO RELATED APPLICATIONS

This application claims the benefit of, and for the purposes of the United States of America the benefit under 35 USC 119 in relation to, U.S. Patent Application No. 63/285,970 filed Dec. 3, 2021, and U.S. Patent Application No. 63/375,526 filed Sep. 13, 2022, the entire disclosures of which are hereby incorporated herein by reference.

### FIELD

This disclosure relates to apparatus and methods for testing cardiac devices such as replacement heart valves (e.g., prosthetic heart valves). The apparatus and methods may be applied, for example, in accelerated wear testing of heart valves at a frequency at or above 200 beats per minute.

### BACKGROUND

Prosthetic heart valves may be implanted to replace heart valves that are not working properly. Failure of a heart valve can be life-threatening. Replacing a failed heart valve involves invasive procedures. Therefore, it is important to make heart valves that are highly reliable.

The human heart beats on the order of once every second. In 25 years, a valve in a person's heart may experience over 700 million cycles. It is challenging to obtain a reliable estimate of the long term durability of a new heart valve design in a reasonable time.

Accelerated wear testing may be used to cycle a replacement heart valve through a large number of cycles in a reasonable time. In order to achieve these huge cycle numbers in a reasonable time the cycle rate may be set to 1200 cycles per minute or more. The standard requires that a certain proportion of each cycle (e.g., 5%) be at or above a specified pressure differential across the replacement heart valve measured when the valve is closed.

Standards for accelerated wear testing of replacement heart valves include the ISO 5840 standard which calls for 200 million cycles of testing. The current version of ISO 5840 is ISO 5840-1, 2, 3:2021.

Examples of accelerated testing apparatus and methods are described in the following U.S. patent and application Ser. No. 11/109,973, U.S. Pat. No. 10,350,069, 9,662,211, 9,237,935, 9,186,224, 8,627,708, 8,584,538, 8,490,504, and 20110132073A1.

There is a need for methods and apparatus for testing heart valves that are reliable and operate according to desired testing protocols. There is also a need for methods and apparatus capable of executing new testing protocols that may provide enhanced information about the long term reliability of heart valves being tested.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides an apparatus for testing prosthetic valves. The apparatus comprises: a test chamber divided into an inflow chamber and an outflow chamber, the test chamber having a fluid connection path between the inflow chamber and the outflow chamber; a mount for supporting a prosthetic valve under test in the fluid connection path with the prosthetic valve oriented to open to pass flow on the fluid connection path from the inflow chamber toward the outflow chamber and to close to restrict flow on the fluid connection path toward the inflow chamber from the outflow chamber; a fluid driving device operable to vary a pressure differential of a fluid between the inflow chamber and the outflow chamber in repeating cycles which include an open phase in which the valve under test is open and a closed phase in which the valve under test is closed; and wherein the fluid driving device is configured to drive the fluid into the outflow chamber to increase a pressure in the outflow chamber in the closed phase and to drive the fluid out of the outflow chamber to decrease the pressure in the outflow chamber in the open phase.

In some embodiments, the apparatus comprises a fluid substantially filling the inflow chamber and the outflow chamber.

In some embodiments, the fluid is transparent.

In some embodiments, the fluid driving device varies a pressure differential in repeating cycles at least 200 times per minute.

In some embodiments, a source of fluid at the inflow chamber maintains a mean fluid pressure in the inflow chamber.

In some embodiments, the source of fluid comprises a reservoir and a conduit fluidly connecting the reservoir to the inflow chamber.

In some embodiments, the source of fluid comprises a fluid reservoir pressurized with a weight.

In some embodiments, an end point of the conduit in the inflow chamber is not more than 40 mm from the mount.

In some embodiments, the reservoir is configured to contain fluid with a surface elevation of the fluid at least 60 mm higher than the mount.

In some embodiments, the surface elevation does not exceed 500 mm above the mount.

In some embodiments, the surface elevation is in the range of 60 mm to 1.5 m above the mount.

In some embodiments, the reservoir comprises indicia marking the surface elevation.

In some embodiments, the indicia comprise a low fluid level mark and a high fluid level mark.

In some embodiments, an elevation difference between the low fluid level mark and the high fluid level mark is in the range of 10 mm to 70 mm.

In some embodiments, the apparatus may comprise a return tank fluidly connected to the reservoir by a supply line and a return line; wherein the reservoir has a first cell, at least one second cell, and a third cell, with a first barrier between the first cell and the at least one second cell and a second barrier between the at least one second cell and the third cell; wherein the supply line is connected to the first cell, the conduit is connected to one of the at least one second cells, and the return line is connected to the third cell; wherein a pump is configured to move fluid from the return tank to the first cell via the supply line; and wherein the first and second barrier are configured to allow fluid to spill from the first cell to the at least one second cell over the first barrier and from the at least one second cell to the third cell over the second barrier.

In some embodiments, the conduit has a diameter of 5 mm.

In some embodiments, the test chamber has a first end that defines a wall of the inflow chamber, a second end that defines a wall of the outflow chamber and sides extending between the first and second ends and wherein the fluid driving device is fluidly coupled to the inflow chamber by an inflow channel connected through a part of the sides into the inflow chamber.

In some embodiments, the fluid driving device cyclically drives the fluid in or out of the inflow chamber through the inflow channel.

In some embodiments, the fluid driving device is fluidly coupled to the outflow chamber by an outflow channel connected through a part of the sides into the outflow chamber.

In some embodiments, the fluid driving device comprises a reciprocating piston.

In some embodiments, the reciprocating piston is operative to push fluid into the outflow chamber to increase the pressure in the outflow chamber and pull fluid out of the outflow chamber to decrease the pressure in the outflow chamber.

In some embodiments, the fluid driving device comprises a reciprocating bellows.

In some embodiments, the reciprocating bellows is operative to push fluid into the outflow chamber to increase the pressure in the outflow chamber and pull fluid out of the outflow chamber to decrease the pressure in the outflow chamber.

In some embodiments, all or a portion of the walls of the test chamber are transparent to allow visual inspection of the prosthetic valve under test during operation of the apparatus.

In some embodiments, the apparatus may comprise one or more of: a first camera mount configured to support a first camera facing a first end of the test chamber wherein the first end of the chamber is closed by a first cap and wherein the first cap is optically transparent; a second camera mount configured to support a second camera facing a second end of the test chamber wherein the second end of the chamber is closed by a second cap and wherein the second cap is transparent; and a third, fourth and fifth camera mount respectively configured to support a third fourth and fifth camera to view the prosthetic valve through transparent portions of a side wall of the test chamber.

In some embodiments, one or more of the first, second, third, fourth and fifth camera mounts have a camera mounted on them.

In some embodiments, first and second ends of the test chamber are closed by caps and the caps are optically transparent.

In some embodiments, the apparatus comprises plural camera mounts for supporting cameras to view the prosthetic valve from different viewpoints through walls of the test chamber.

In some embodiments, one or both of an outer surface of the wall of the outflow chamber and an inner surface of the wall of the outflow chamber has one or more flat areas through which a valve under test inside the test chamber may be viewed. In some embodiments, the apparatus comprises an adjustable bypass valve connected to allow fluid flow between the outflow chamber and the inflow chamber to control the pressure of the outflow chamber.

In some embodiments, the apparatus comprises a second non-adjustable bypass valve connected to allow fluid flow from the outflow chamber to the inflow chamber.

In some embodiments, the apparatus comprises: a first sensor operative to measure a pressure of the fluid in the inflow chamber; a second sensor operative to measure a pressure of the fluid in the outflow chamber; and a controller configured to store an upper control limit and a lower control limit, determine a target variable value based at least on output signals from the first and second sensors, and in response to a relationship of the target variable value to the upper and lower control limit control an actuator to incrementally open or close a bypass valve so as to cause the target variable value to be varied to increase the target variable value if the target variable value is below the lower control limit or to decrease the target variable value if the target variable value is above the upper control limit.

In some embodiments, the apparatus comprises a controller configured to receive sensor data indicating a differential pressure between the inflow chamber and the outflow chamber, process the sensor data and to assess whether or not in each cycle of the fluid driving device passed requirements for peak differential pressure and high pressure time, compute a target variable wherein the target variable is a rolling cycle passing rate, and control a bypass valve based on the rolling cycle passing rate to maintain the rolling cycle passing rate between a lower control limit and an upper control limit.

In some embodiments, the rolling cycle passing rate is determined for a window containing passing information for at least the most recent 900 cycles.

In some embodiments, the target variable value is controlled to be at or slightly above the lower control limit.

In some embodiments, the bypass valve is a plug valve.

In some embodiments, the bypass valve comprises a plug having an aperture that has a circular cross section.

In some embodiments, the bypass valve comprises a plug having an aperture that has a rectangular cross section.

In some embodiments, a plug of the plug valve has first and second axial end portions that are smaller in diameter than a portion of the plug between the first and second axial end portions.

In some embodiments, the first axial end portion is circumscribed by a collar and the plug valve includes a seal between the collar and the first axial end portion.

In some embodiments, the seal comprises an O-ring.

In some embodiments, the second axial end is circumscribed by an O-ring.

In some embodiments, the O-ring has a hardness of between 60 and 80 on the Shore A Hardness Scale.

In some embodiments, the O-ring is made of silicone.

In some embodiments, the apparatus comprises an encoder connected to measure a degree of rotation of the plug valve and transmit a signal indicating the degree of rotation to the controller.

In some embodiments, the apparatus comprises a user interface operative to receive user input specifying a value for one or both of the upper control limit and the lower control limit.

In some embodiments, the apparatus comprises at least one compliance element in the inflow or outflow chamber.

In some embodiments, the compliance element comprises a soft tube filled with a compressible gas.

In some embodiments, the soft tube comprises a wall made of silicone.

In some embodiments, the compressible gas is air.

In some embodiments, a material of a wall of the compliance element has a hardness in the range of SHORE A 30 and 40.

In some embodiments, the soft tube is arranged in a circular, arc, spiral or straight configuration.

In some embodiments, ends of the soft tube are connected to form a ring.

In some embodiments, ends of the soft tube are plugged.

In some embodiments, the soft tube is inserted into a groove in a wall of the test chamber.

In some embodiments, the at least one compliance element comprises a plurality of rings of the soft tube extending around the opening between the inflow chamber and the outflow chamber.

In some embodiments, the soft tube has an external diameter in the range of 5 mm to 30 mm.

In some embodiments, the soft tube has an external diameter in the range of 7 mm to 9 mm.

In some embodiments, the soft tube has a wall thickness in the range of 0.5 mm to 1.5 mm.

In some embodiments, the soft tube has a wall thickness of about 1 mm.

In some embodiments, the at least one compliance element comprises a plurality of the soft tubes.

In some embodiments, the soft tubes are removable.

In some embodiments, a total sealed volume of the compliance elements installed in the inflow chamber is in the range of 15 ml to 240 ml.

In some embodiments, a total sealed volume of the compliance elements installed in the outflow chamber is in the range of 0 ml to 240 ml.

In some embodiments, the soft tubes are held in place in the test chamber by one or more holding clips.

In some embodiments, the soft tubes are held in place in the test chamber by a plurality of holding clips, each of the holding clips having an opening smaller than a diameter of the soft tubes.

In some embodiments, the apparatus comprises at least one of the soft tubes in the inflow chamber in proximity to the prosthetic valve under test by not more than 100 mm.

Another aspect of the invention provides an apparatus for testing prosthetic valves comprising: a test chamber divided into an inflow chamber and an outflow chamber, the test chamber having a fluid connection path between the inflow chamber and the outflow chamber; a mount for supporting a prosthetic valve under test in the fluid connection path; a fluid driving device operable to vary a pressure differential of a fluid between the inflow chamber and the outflow chamber in repeating cycles; and a source of fluid at the inflow chamber that maintains a mean fluid pressure in the inflow chamber.

In some embodiments, the source of fluid comprises a reservoir and a conduit fluidly connecting the reservoir to the inflow chamber.

In some embodiments, the reservoir is pressurized with a weight.

In some embodiments, an end point of the conduit in the inflow chamber is not more than 40 mm from the mount.

In some embodiments, the reservoir is configured to contain fluid with a surface elevation of the fluid at least 60 mm higher than the mount.

In some embodiments, the surface elevation does not exceed 500 mm above the mount.

In some embodiments, the surface elevation is in the range of 60 mm to 1.5 m above the mount.

In some embodiments, the reservoir comprises indicia marking the surface elevation.

In some embodiments, the indicia comprise a low fluid level mark and a high fluid level mark.

In some embodiments, an elevation difference between the low fluid level mark and the high fluid level mark is in the range of 10 mm to 70 mm.

In some embodiments, the apparatus may comprise a return tank fluidly connected to the reservoir by a supply line and a return line; wherein the reservoir has a first cell, at least one second cell, and a third cell, with a first barrier between the first cell and the at least one second cell and a second barrier between the at least one second cell and the third cell; wherein the supply line is connected to the first cell, the conduit is connected to one of the at least one second cells, and the return line is connected to the third cell; wherein a pump is configured to move fluid from the return tank to the first cell via the supply line; and wherein the first and second barrier are configured to allow fluid to spill from the first cell to the at least one second cell over the first barrier and from the at least one second cell to the third cell over the second barrier.

In some embodiments, at least a portion of a wall of the reservoir is transparent or translucent to allow observation of a level of fluid in the reservoir.

In some embodiments, the conduit has a diameter of 5 mm or less.

Another aspect of the invention provides a method for testing a prosthetic valve comprising the steps of: while testing a prosthetic valve, calculating a mean fluid pressure in an inflow or outflow chamber and recording the mean fluid pressure as a reference fluid pressure; continuously monitoring a current mean fluid pressure in the inflow or outflow chamber and determining a difference between the current mean fluid pressure and the reference fluid pressure; and if the difference between the current mean fluid pressure and the reference fluid pressure exceeds a pre-set value, send or display a warning or error message.

In some embodiments, the method comprises calculating the current mean fluid pressure based on a previous N test cycles where N is an integer greater than 1.

Another aspect of the invention provides an apparatus for testing prosthetic valves comprising: a test chamber divided into an inflow chamber and an outflow chamber, the test chamber having a fluid connection path between the inflow chamber and the outflow chamber; a mount for supporting a prosthetic valve under test in the fluid connection path with the prosthetic valve oriented to open to pass flow on the fluid connection path from the inflow chamber toward the outflow chamber and to close to restrict flow on the fluid connection path toward the inflow chamber from the outflow chamber; a fluid driving device operable to vary a pressure differential of a fluid between the inflow chamber and the outflow chamber in repeating cycles which include an open phase in which a valve under test is open and a closed phase in which the prosthetic valve under test is closed; and at least one compliance element in the form of a soft tube installed in the inflow or outflow chamber for providing compliance to smooth a pressure waveform in the inflow or outflow chamber.

In some embodiments, the soft tube is filled with a compressible gas.

In some embodiments, the soft tube comprises a wall made of silicone.

In some embodiments, the compressible gas is air.

In some embodiments, a material of a wall of the compliance element has a hardness in the range of SHORE A 30 and 40.

In some embodiments, the soft tube is arranged in a circular, arc, spiral or straight configuration.

In some embodiments, ends of the soft tube are connected to form a ring.

In some embodiments, ends of the soft tube are plugged.

In some embodiments, the soft tube is inserted into a groove in a wall of the test chamber.

In some embodiments, the at least one compliance element comprises a plurality of rings of the soft tube extending around the opening between the inflow chamber and the outflow chamber.

In some embodiments, the soft tube has an external diameter in the range of 5 mm to 30 mm.

In some embodiments, the soft tube has an external diameter in the range of 7 mm to 9 mm.

In some embodiments, the soft tube has a wall thickness in the range of 0.5 mm to 1.5 mm.

In some embodiments, the soft tube has a wall thickness of about 1 mm.

In some embodiments, the at least one compliance element comprises a plurality of the soft tubes.

In some embodiments, the soft tubes are removable.

In some embodiments, a total sealed volume of the at least one compliance elements installed in the inflow chamber is in the range of 15 ml to 240 ml.

In some embodiments, a total sealed volume of the at least one compliance elements installed in the outflow chamber is in the range of 0 ml to 240 ml.

In some embodiments, the soft tubes are held in place in the test chamber by one or more holding clips.

In some embodiments, the soft tubes are held in place in the test chamber by a plurality of holding clips, each of the holding clips having an opening smaller than a diameter of the soft tubes.

In some embodiments, at least one of the soft tubes in the inflow chamber is in proximity to the prosthetic valve under test by not more than 100 mm.

Another aspect of the invention provides an apparatus for testing prosthetic valves comprising: a test chamber divided into an inflow chamber and an outflow chamber, the test chamber having a fluid connection path between the inflow chamber and the outflow chamber; a mount to support a prosthetic valve in the fluid connection path; a fluid driving device operable to vary a pressure differential of a fluid between the inflow chamber and the outflow chamber in repeating cycles; a bypass valve comprising a variable aperture arranged to allow fluid to flow between the outflow chamber and the inflow chamber; a first sensor operative to measure a pressure of the fluid in the inflow chamber; a second sensor operative to measure a pressure of the fluid in the outflow chamber; a control system configured to: store an upper control limit and a lower control limit, determine a value for a target variable based at least on output signals from the first and second sensors, and in response to a relationship of the target variable value to the upper and lower control limits, control an actuator to incrementally open or close the bypass valve so as to cause the target variable value to be varied to increase the target variable value if the target variable value is below the lower control limit or to decrease the target variable value if the target variable value is above the upper control limit.

In some embodiments, the target variable is a peak differential pressure across the prosthetic valve.

In some embodiments, the target variable is a high pressure time for the differential pressure across the prosthetic valve.

In some embodiments, the target variable is a cycle passing rate for the prosthetic valve.

In some embodiments, the cycle passing rate is calculated based on a plurality of past test cycles.

In some embodiments, the target variable value is controlled to be at a midpoint between the upper control limit and the lower control limit.

In some embodiments, the target variable value is controlled to be at or slightly above the lower control limit.

In some embodiments, the target variable value is controlled to be between the lower control limit and the mean of the upper and lower control limits.

In some embodiments, the actuator comprises a servomotor or a stepper motor or a linear actuator that is mechanically coupled to operate the bypass valve.

In some embodiments, the bypass valve is a plug valve.

In some embodiments, the bypass valve comprises a plug having an aperture which has a circular cross section.

In some embodiments, the bypass valve comprises a plug having an aperture that has a rectangular cross section.

In some embodiments, a plug of the plug valve has first and second axial end portions that are smaller in diameter than a portion of the plug between the first and second axial end portions.

In some embodiments, the first and second axial end portions are tapered.

In some embodiments, the first axial end portion is circumscribed by a collar and the plug valve includes a seal between the collar and the first axial end portion.

In some embodiments, the seal comprises an O-ring.

In some embodiments, the second axial end is circumscribed by an O-ring.

In some embodiments, the O-ring has a hardness of between 60 and 80 on the Shore A Hardness Scale.

In some embodiments, the O-ring is made of silicone.

In some embodiments, the apparatus comprises an encoder connected to measure the degree of rotation of the plug valve and transmit a signal indicating a degree of rotation to the control system.

In some embodiments, the apparatus comprises a user interface operative to receive a user input specifying a value for one or both of the upper control limit and the lower control limit.

Another aspect of the invention provides an apparatus for testing prosthetic valves comprising: a test chamber divided into an inflow chamber and an outflow chamber, the test chamber having a fluid connection path between the inflow chamber and the outflow chamber; a mount for supporting a prosthetic valve under test in the fluid connection path; a fluid driving device operable to vary a pressure differential of a fluid between the inflow chamber and the outflow chamber; a bypass valve arranged to allow fluid to flow between the outflow chamber and the inflow chamber; a controller connected to control the bypass valve; wherein the controller is configured to: receive sensor data indicating a fluid pressure in both the inflow chamber and the outflow chamber; process the sensor data to assess whether or not each cycle of the fluid driving device passed requirements for peak differential pressure and high pressure time; compute a rolling cycle passing rate; and control the bypass valve based on the rolling cycle passing rate to maintain the rolling cycle passing rate between a lower control limit and an upper control limit.

In some embodiments, the upper control limit is less than 100%.

In some embodiments, the upper control limit is greater than 99%.

In some embodiments, the lower control limit is equal to or greater than 95%.

In some embodiments, the controller controls the bypass valve to maintain the rolling cycle passing rate incrementally above the lower control limit.

In some embodiments, the rolling cycle passing rate is determined for a window containing passing information for at least the most recent 900 cycles.

Another aspect of the invention provides a method for modulating testing characteristics of a prosthetic valve testing apparatus, the method comprising: providing a value for one or both of an upper control limit and a lower control limit corresponding to a target variable to a controller; measuring values for a pressure differential between an inflow chamber and an outflow chamber; computing a target variable value based on the pressure differential values; and based on the target variable value, controlling an actuator to modulate a bypass valve positioned between the inflow and outflow chamber to keep the target variable value in a range defined by the lower and upper control limits. In some embodiments, the method comprises rotating the bypass valve with the actuator wherein the bypass valve is a plug valve.

In some embodiments, the method comprises measuring a degree of rotation of the plug valve.

In some embodiments, the method comprises rotating the bypass valve with the actuator wherein the actuator is a servomotor or a stepper motor or a linear actuator.

In some embodiments, the method comprises maintaining the target variable value at or incrementally above the lower control limit.

In some embodiments, the method comprises opening the bypass valve at a beginning of a test of the prosthetic valve, and then incrementally closing the bypass valve to a point sufficient to bring the target variable value to be between the upper and lower control limits.

In some embodiments, the method comprises opening the bypass valve sufficiently to bring the target variable value below the lower control limit if the target variable value exceeds the upper control limit.

Another aspect of the invention provides a method for modulating testing characteristics of a prosthetic valve testing apparatus, the method comprising: providing a value for one or both of an upper control limit and a lower control limit corresponding to a target variable to a controller; measuring values for a pressure in an inflow chamber and an outflow chamber; computing a target variable value based on at least the pressure values; and controlling an actuator to modulate a bypass valve positioned between the inflow chamber and the outflow chamber to reduce the target variable value below the lower control limit if the target variable value exceeds the upper control limit.

In some embodiments, the method comprises storing a position for the actuator that maintains the target variable at or below the lower control limit.

In some embodiments, the method comprises modulating the bypass valve to the stored position when the target variable exceeds the upper control limit.

Another aspect of the invention provides an apparatus for testing prosthetic valves comprising: a test chamber divided into an inflow chamber and an outflow chamber, the test chamber having a fluid connection path between the inflow chamber and the outflow chamber; a mount for supporting a prosthetic valve under test in the fluid connection path; a fluid driving device operable to vary a pressure differential of a fluid between the inflow chamber and the outflow chamber; and wherein all or a portion of walls of the test chamber are transparent to allow visual inspection of the prosthetic valve.

In some embodiments, the wall of the test chamber containing the fluid driving device is not transparent.

In some embodiments, the apparatus comprises one or more of: a first camera mount configured to support a first camera facing a first end of the test chamber wherein the first end of the test chamber is closed by a first cap and the first cap is optically transparent; a second camera mount configured to support a second camera facing a second end of the test chamber wherein the second end of the test chamber is closed by a second cap and the second cap is optically transparent; and a third, fourth and fifth camera mount respectively configured to support a third, a fourth and a fifth camera arranged to view the prosthetic valve through transparent portions of a side wall of the test chamber.

In some embodiments, one or more of the first, second, third, fourth and fifth camera mounts have a camera mounted on them.

In some embodiments, the test chamber allows for visual inspection of the prosthetic valve under test from all sides of the test chamber except for the side containing the fluid driving device.

Further aspects and example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 is a schematic vertical cross-section of an example valve-testing apparatus.

FIG. 2 and FIG. 3 illustrate fluid flow in phases of operation of the apparatus of FIG. 1.

FIG. 4 is a flow chart for an example method of controlling a bypass valve.

FIG. 5 is a schematic illustration of an example valve testing apparatus incorporating bypass valve control.

FIG. 6A and FIG. 6B are cross section views of an example bypass valve in an open and closed position respectively.

FIG. 7A is a cross section view of an example plug-type bypass valve having a circular aperture in an open configuration and schematically illustrated control apparatus.

FIG. 7B is a cross section view of an example plug-type bypass valve having a rectangular aperture in an open configuration and schematically illustrated control apparatus.

FIG. 7C is a cross section view of an example plug-type bypass valve in a closed configuration and schematically illustrated control apparatus.

FIG. 8 and FIG. 9 are example cross-sections for a wall of an outflow chamber of a testing apparatus such as the valve testing apparatus of FIG. 1.

FIG. 10 is a graph showing example system pressure signals as functions of time.

FIG. 11 is a flow chart that illustrates operation of an example automatic pressure verification system.

FIG. 12 is a schematic cross-section of another example valve-testing apparatus.

FIG. 13 is a schematic cross-section of a test chamber for a cardiovascular device that includes inflow and outflow compliance elements.

FIG. 14 is a schematic plan view of an example arrangement of tubular compliance elements.

FIG. 15 is a schematic elevation view of an example pressure maintenance system.

FIG. 16A is a schematic cross-section of another example valve-testing apparatus.

FIG. 16B is a schematic cross-section of another example valve-testing apparatus.

FIG. 17 is a schematic elevation view of an example pressure maintenance system.

FIG. 18 is a top plan view of a fluid reservoir of the pressure maintenance system of FIG. 17.

FIG. 19, FIG. 20 and FIG. 21 are schematic views that illustrate example pressure maintenance systems that use weighted pistons to apply and maintain a desired fluid pressure.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

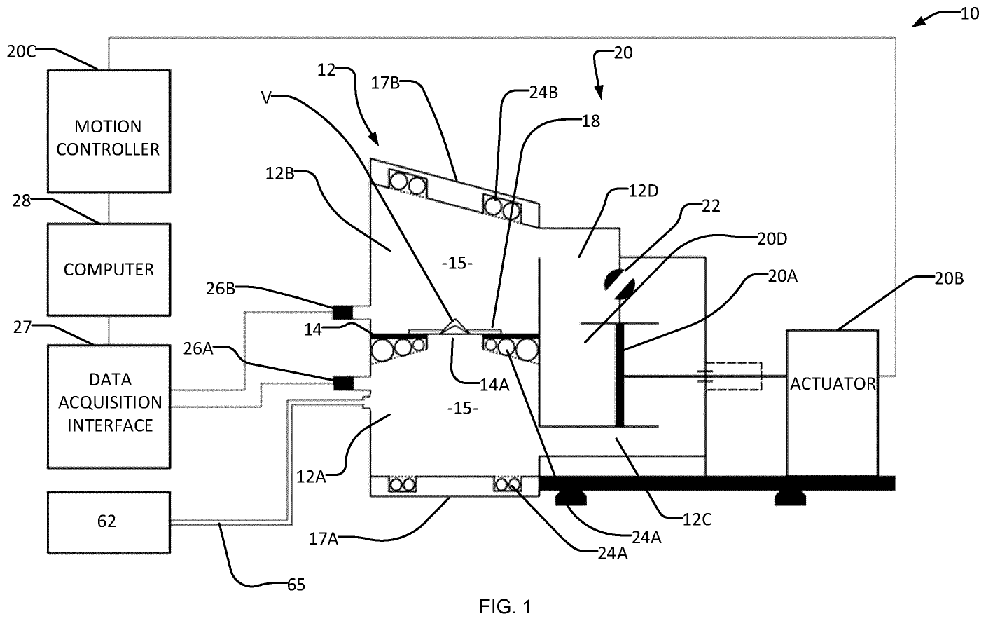

FIG. 1 is a vertical cross section of apparatus 10 according to an example embodiment. Apparatus 10 comprises a testing chamber 12 divided into an inflow chamber 12A and an outflow chamber 12B. Chamber 12 may be round in cross section such that inflow chamber 12A and outflow chamber 12B are at least generally cylindrical. In the illustrated embodiment, test chamber 12 is closed by end caps 17A and 17B and a partition 14 separates inflow chamber 12A and outflow chamber 12B

Test chamber 12 is filled with a fluid 15 (e.g., a saline solution or another blood analog fluid). In apparatus 10 of FIG. 1, a static pressure of fluid 15 is maintained at a desired reference pressure by reservoir 62.

A valve V under test is mounted to a mount 18 at an aperture 14A that connects between inflow chamber 12A and an outflow chamber 12B. Mount 18 is configured to hold valve V and may be removably affixed to allow valve V to be positioned with one side contacting fluid 15 in outflow chamber 12B and one side contacting fluid 15 in inflow chamber 12A. For example, mount 18 may be removably affixed to partition 14 in any suitable manner 18A (e.g., clips, screws (as in FIG. 14), magnets, clamps, etc.).

Aperture 14A is a circular aperture in some embodiments. Valve V is oriented so that valve V opens when pressure at the side of valve V facing inflow chamber 12A exceeds the pressure at the side of valve V facing outflow chamber 12B by a sufficient amount and so that valve V closes when the pressure on the side of valve V facing outflow chamber 12B exceeds the pressure at the side of valve V facing inflow chamber 12A by a sufficient amount.

In some embodiments the walls of inflow chamber 12A and outflow chamber 12B including end caps 17A and 17B are made of a material that is translucent or transparent to facilitate observation of valve V. Apparatus 10 may be constructed such that valve V can be observed operating and images and/or video of valve V may be acquired from outside of test chamber 12 from multiple viewing directions including one or more views of the front side of valve V and one or more views of the rear side of valve V while valve V is being tested. Arranging apparatus 10 so that clear views of the back side of valve V can be obtained allows collection of images that reveal details about how valve V moves and deforms as it operates.

In some embodiments, the top wall of testing chamber 12B (in the illustrated example end cap 17B) provides a surface that is sloped so that any air bubbles that may find their way into testing chamber 12 are carried by buoyant forces to a collection zone which is located so that any accumulated air does not interfere with operation of apparatus 10 or observation of a valve under test and/or equipped with a port through which any collected air may be removed from test chamber 12. For example, in the illustrated example, end cap 17B is inclined at an angle to horizontal (the angle only needs to be steep enough to cause bubbles to rise to a high point where the air can accumulate and/or be removed; an angle of a few degrees, such as about 8 degrees may suffice for this purpose).

Apparatus 10 includes a driving mechanism 20 that operates to cycle valve V. In the illustrated embodiment, driving mechanism 20 includes a reciprocating member shown in FIG. 1 as a piston 20A that is driven to reciprocate by an actuator 20B controlled by a motion controller 20C. The reciprocating member may, for example, comprise a reciprocating piston, bellows, or diaphragm.

Dimensions of the reciprocating member and a travel (stroke) of the reciprocating member may be selected to achieve desired test conditions for valve V. In some embodiments, the reciprocating member has an average diameter in the range of about 40 mm to about 100 mm. In some embodiments the reciprocating member has a stroke in the range of about 5 mm to about 20 mm.

The displacement and velocity of piston 20A may be controlled by motion controller 20C. In some embodiments motion controller 20C is configured to control piston 20A to move at different velocities on forward and reverse parts of a reciprocation cycle. In some embodiments motion controller 20C is configured to control piston 20A to have velocities that vary in any desired manner over a reciprocation cycle. In some embodiments, motion controller 20C controls velocity and/or acceleration of piston 20A as a function of position of piston 20A. The control exerted by motion controller 20C may be manifested as a waveform applied by motion controller 20C to actuator 20B. Actuator 20B may, for example, comprise a motor. In some embodiments actuator 20B comprises a servomotor. In some embodiments actuator 20B comprises a linear actuator.

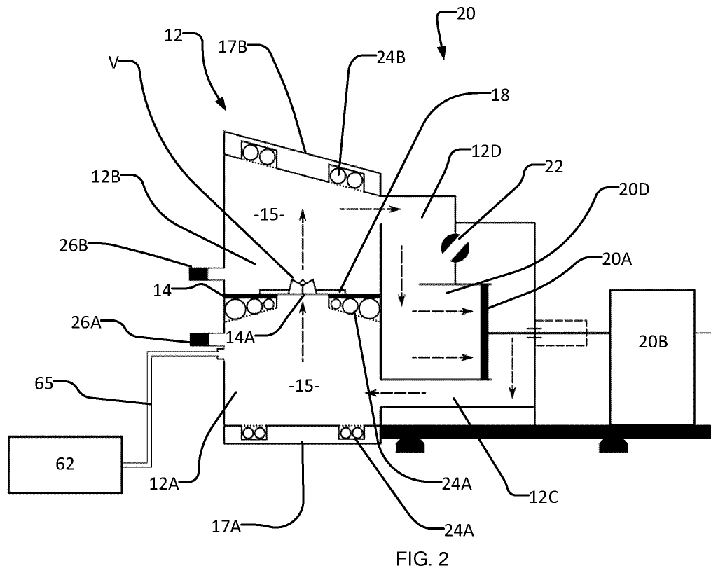

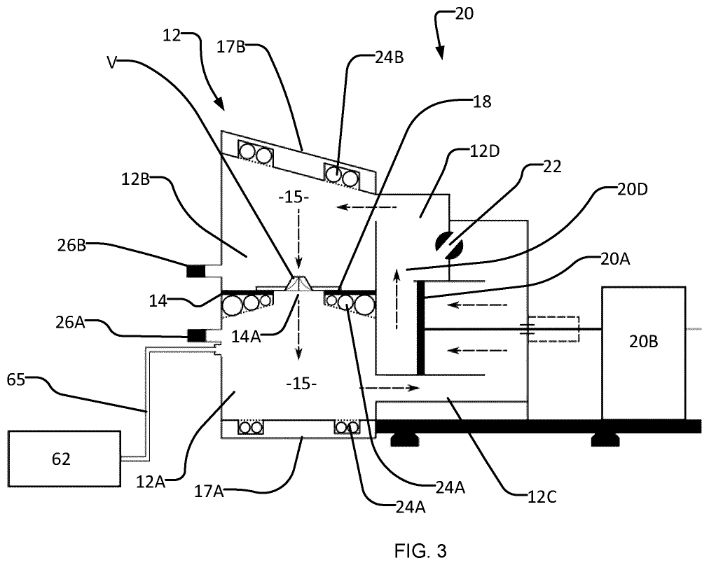

Reciprocation of piston 20A causes fluid 15 to circulate around a flow loop 20D. An example testing cycle has two phases. These are illustrated in FIG. 2 and FIG. 3. In a first phase as shown in FIG. 2, piston 20A is moved in a direction such that fluid 15 is withdrawn from outflow chamber 12B and delivered into inflow chamber 12A. As piston 20A moves in this direction (backwards—to the right in FIG. 1 and FIG. 2), the motion of piston 20A increases the volume of outflow path 12D so that fluid 15 is drawn out of outflow chamber 12B causing the pressure in outflow chamber 12B to decrease. This decreases the differential pressure between in inflow chamber 12A and outflow chamber 12B across valve V. When the differential pressure decreases below zero to a negative level (i.e., where pressure of fluid 15 in inflow chamber 12A adjacent to valve V exceeds pressure of fluid in outflow chamber 12B at valve V), the differential pressure causes valve V to open, allowing fluid 15 to pass through valve V to outflow chamber 12B. If valve V was previously closed, the fluid is forced through valve V and valve V opens as a result of the pressure differential across valve V.

In a second phase as shown in FIG. 3, the direction of motion of piston 20A is reversed (i.e., so that piston 20A travels in the forward direction—to the left in FIG. 1 and FIG. 3). When piston 20A is moving in this direction the volume of outflow path 12D is reduced and the pressure of fluid 15 in outflow path 12D is increased. Fluid 15 is delivered through outflow path 12D into outflow chamber 12B. This causes an increase in pressure of outflow chamber 12B and an increase of the differential pressure between in inflow chamber 12A and outflow chamber 12B across valve V.

When the differential pressure between inflow chamber 12A and outflow chamber 12B across valve V increases above zero to a positive level, the differential pressure then causes valve V to close.

After valve V is closed during the second phase, when piston 20A continues to move in the same direction, the volume of outflow path 12D is further reduced so that the pressure of fluid 15 in outflow path 12D and outflow chamber 12B is further increased, and the magnitude of the differential pressure between inflow chamber 12A and outflow chamber 12B across valve V is further increased.

Accelerated wear testing standards for cardiac implants (e.g., ISO-5840 standards) require that the differential pressure across valve V must reach at least a stated value (e.g., for valves of some types, 100 mmHg) for at least a stated period of time (e.g., for valves of some types, 5% of the valve open-close cycle time) in each valve cycle. On the other hand, it can be undesirable to expose valve V to pressure differentials beyond the requirements of the applicable standard or to expose valve V to pressure differentials for periods of time in excess of the time period required by the applicable standard.

Furthermore, over the course of a test, the pressure across valve V at a particular phase of the cycle may fluctuate. Pressure differentials across valve V at different phases of the testing cycle can change over time for a number of reasons, including, but not limited to, wear across valve V leading to reduced seal capability when valve Vis closed, leakage of air from the apparatus, and/or leakage of air from other elements in the apparatus.

Apparatus 10 may include a bypass valve 22 that is operable to modulate hydraulic characteristics across valve V and in particular to manage differential pressures across a prosthetic valve under test. In some embodiments bypass valve 22 is actively controlled.

Incrementally opening bypass valve 22 permits fluid to pass more easily through bypass valve 22, thereby reducing the magnitude of differential pressures across valve V. Incrementally closing bypass valve 22 causes bypass valve 22 to present more resistance to the flow of fluid through bypass valve 22, thereby increasing the magnitude of differential pressures across valve V.

The degree of opening of bypass valve 22 may be controlled so that during testing the magnitude of differential pressure across a prosthetic valve under test reaches at least a value required by the applicable standard and also maintains at least a certain magnitude of differential pressure for a length of time (“high pressure time”) in each cycle as required by the applicable standard. The control methodology may also be selected to keep the magnitude of differential pressure from exceeding the value required by the applicable standard by more than a set amount. For example, a control methodology may operate to maintain the peak differential pressure across the valve under test within a range defined by upper and lower bounds wherein the lower bound is set high enough to achieve a pressure profile in each cycle that satisfies the requirements of the applicable standard.

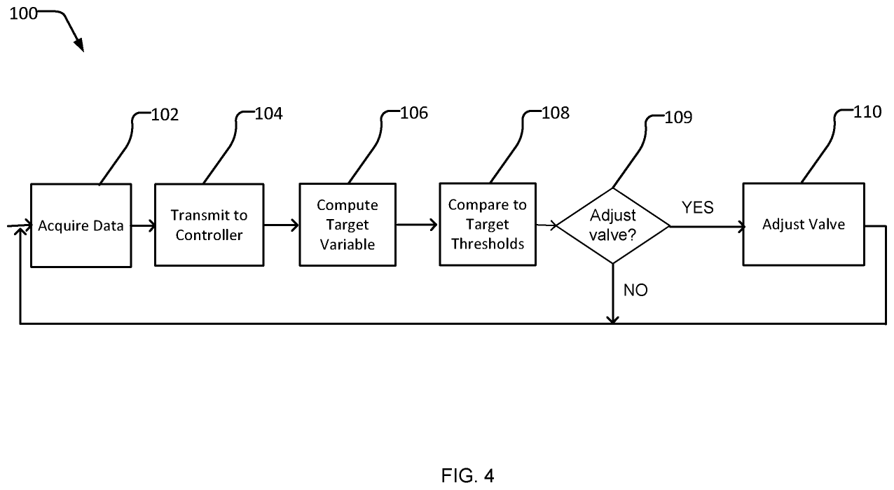

FIG. 4 is a flow chart for a method 100 of operating a bypass valve according to an example embodiment. Step 102 comprises acquiring data from which the differential pressure across a valve under test may be determined. Step 102 may, for example, comprise obtaining pressure measurements for fluid on either side of a valve using sensors. For example, the sensors may include at least one pressure sensor operative to measure pressure in the inflow chamber of the accelerated wear device and at least one pressure sensor operative to measure pressure in the outflow chamber of the accelerated wear device. As another example, a differential pressure sensor may be provided that is operative to measure a difference between the pressures in the inflow and outflow chambers. Step 102 may operate continuously to provide a stream of data.

In step 104 the acquired data is delivered to a control system.

Step 106 comprises computing values for one or more target variables using the data acquired in step 102. The target variable is any quantity that can be calculated using the acquired data from step 102. For example, the target variables may comprise one or more of: peak differential pressure across a prosthetic valve, and high pressure time. The peak differential pressure is the maximum pressure difference between the outflow chamber and the inflow chamber. The high pressure time is the percentage of time during a test cycle in which the pressure difference sustained across the valve under test exceeds a specified value.

Step 106 may also maintain a record of the cycle passing rate. The cycle passing rate is the percentage of cycles that achieve the required pressure differential across the prosthetic valve and also have at least the required high pressure time. The peak differential pressure and high pressure time may be prescribed by a testing standard such as ISO-5840. In some embodiments the cycle passing rate is used as a target variable.

Steps 108 and 110 operate in a way that attempts to maintain the value of a target variable between upper and lower bounds. For example, steps 108 and 110 may operate to attempt to:
maintain the peak differential pressure across a valve V under test in each cycle above a lower bound selected such that if the peak differential pressure is below the lower bound then the cycle would not “pass” because one or both of the peak differential pressure or the high pressure time does not meet the applicable requirements. The upper bound may be set somewhat higher than the lower bound;
maintain the high pressure time in each cycle above a lower bound selected to match or slightly exceed the minimum required high pressure time. The upper bound may be set somewhat higher than the lower bound; and/or
maintain the cycle passing rate between a lower bound and an upper bound (in some embodiments the upper bound is set to be less than 100%).

For example, step 108 may comprise comparing the target variable(s) to the corresponding upper and lower bounds, a target set point and/or corresponding upper and lower target control limits.

Step 109 determines if the results of step 108 indicate that bypass valve 22 should be adjusted. If YES then method 100 proceeds to step 110 otherwise (NO result at step 109) method 100 returns to step 102.

Step 110 comprises adjusting a bypass valve 22 so that, when bypass valve 22 is fully or partially open, fluid can flow between an outflow chamber and an inflow chamber. The rate of fluid flow through bypass valve 22 for a given pressure differential across bypass valve 22 may be varied by changing the degree to which bypass valve 22 is open. In some embodiments, step 110 is configured to open or close bypass valve 22 in a small increment each time step 110 is repeated.

Method 100 controls the degree of opening of bypass valve 22 based on the measurements obtained in step 102 to keep the target variable calculated in step 106 in the range between the corresponding upper and lower bounds.

In some embodiments, bypass valve 22 is adjusted using a proportional control scheme. In such embodiments, if the target variable value computed in step 106 is not equal to a set point for the target variable or lies outside of a range defined by upper and lower control limits for the target variable in step 108, the opening of bypass valve 22 is adjusted in step 110 by an amount that is proportional to the difference between the current value of the target variable and the corresponding set point or the closest one of the upper and lower control limits or a value within the target range (e.g. a midpoint of the target range).

In some embodiments, if the target variable computed in step 106 lies within a target range (e.g., in a range between upper and lower control limits) in step 108, bypass valve 22 is not adjusted in step 110.

In some embodiments, bypass valve 22 is automatically adjusted by a control system that employs two or more of proportional control, integral control, and derivative control algorithms. Depending on the value of the target variable and its trend over time relative to upper and lower control limits, bypass valve 22 may be automatically adjusted so that the target variable stays within the range of the upper and lower control limit. Proportional control, and control schemes employing two or more of proportional control, integral control, and derivative control are particularly advantageous for controlling the peak differential pressure across the prosthetic valve.

In some embodiments, bypass valve 22 is adjusted using an incremental control scheme in which, as long as the target variable value is within the range defined between upper and lower control limits, bypass valve 22 is not adjusted. If the value of the target variable is below the lower control limit then bypass valve 22 is incrementally adjusted (to incrementally open or close bypass valve 22) in a direction that tends to cause the target variable value to increase. If the value of the target variable is above the upper control limit then bypass valve 22 is incrementally adjusted (to incrementally open or close bypass valve 22) in a direction that tends to cause the target variable value to decrease.

This method of control can be used to control target variables such as maximum pressure differential across valve V and/or high pressure time to be at least equal to required values and also to not unnecessarily exceed the required values.

It can be desirable to maintain a target variable value such as high pressure time, maximum magnitude of pressure differential or cycle passing rate within the range between upper and lower control limits. At the same time, it can be further desirable to keep a target variable closer to the one of upper and lower control limits which, if crossed would result in cycles that do not pass. For example, it may be desirable to maintain a value of a target variable such as high pressure time, maximum magnitude of pressure differential or cycle passing rate to be slightly above a lower control limit.

In some embodiments, if the target variable value exceeds the upper control limit, bypass valve 22 is adjusted by an increment (“upper increment”) that is different from (and in the opposite direction to) the increment (“lower increment”) by which bypass valve is adjusted when the target variable is below the lower control limit. In the following example it is desired to maintain the value of a target variable close to a lower control limit. In this example, the upper increment is larger than the lower increment. In some embodiments the upper increment is large enough to cause the target variable value to decrease from being above the upper control limit to being below the lower control limit. In some embodiments a setting for bypass valve 22 that has been previously determined to cause the value of the target variable to be at or close to or just below the lower control limit is stored and the upper increment is selected to set the opening of the bypass valve to the stored setting.

In embodiments where the upper increment is large enough to cause the target variable value to fall to below the lower control limit, then in subsequent cycles the above method will alter the setting of the bypass valve by the lower increment until the target variable value is again in the range between the lower and upper control limits.

In some embodiments, the incremental control scheme as described herein is used at the beginning of a test cycle for a prosthetic valve. The apparatus may be initially configured (for example, by setting bypass valve 22 so that it starts in a generally open position) so that the target variable will be below the lower control limit. The incremental control scheme then controls bypass valve 22 by incrementing bypass valve 22 closed until the target variable has a value that lies just within the range between the upper lower control limits (e.g., just above the lower control limit).

In some embodiments, a control algorithm uses a rolling average value of a target variable based on measurements taken over a plurality of cycles. The bypass valve can then be adjusted in response to the rolling average of the target variable to keep the target variable within the range defined by the corresponding upper and lower control limits.

By way of example, such a method may be advantageously used to control bypass valve 22 based on a value of a cycle passing rate. A cycle passes if it complies with the applicable requirements (e.g., for high pressure time and magnitude of pressure differential across valve V). A cycle passing rate may be determined based on measurements made for cycles in a sliding window containing data for a plurality of past test cycles. For each cycle the control scheme determines whether or not the cycle is a passing cycle. The cycle passing rate may be determined for a window containing data for a sufficient number of cycles (e.g., 100 to 50,000 cycles). The cycle passing rate may be periodically updated (e.g., every cycle or every few cycles). The cycle passing rate may, for example, be controlled using an incremental control scheme based on upper and lower control limits as described above. Advantageously the upper control limit is below 100%. For example, an upper control limit for the cycle passing rate may be set to a value slightly less than 100% (e.g., 29,999/30,000).

For example, a cycle passing rate may be determined every cycle based on the most recent N cycles (N may for example be in the range of 200 to 30,000) cycles. If the cycle passing rate departs from a desired value or range then bypass valve 22 may be controlled, for example as described above, to bring the cycle passing rate toward the desired value or to within the desired range.

By way of further example, such a method could also be used to control the peak differential pressure and high pressure time across valve V. A rolling average of the peak differential pressure and high pressure time across valve V may be calculated based on a plurality of past cycles. Bypass valve 22 may then be adjusted in a way that attempts to keep the rolling average target variable value within a desired range (e.g., between upper and lower control limits).

Calculating the target variable based on a rolling average may advantageously reduce the vulnerability of the control scheme to individual anomalous test cycles. This may result in smoother control of the pressure characteristics across valve V.

Bypass valve 22 may be adjusted during, in between, or over the course of one or several cycles.

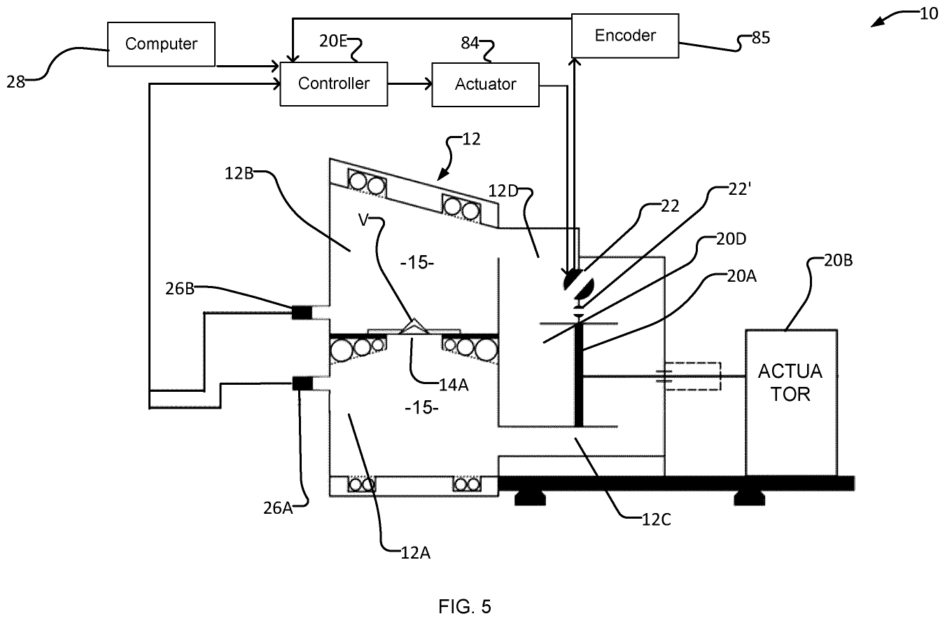

FIG. 5 shows bypass valve 22 in apparatus 10 according to an example embodiment. Bypass valve 22 is connected between inflow path 12C and outflow path 12D such that when bypass valve 22 is open or partially open, fluid 15 can flow between inflow path 12C and outflow path 12D through bypass valve 22.

Pressure sensors 26A and 26B respectively measure pressures in inflow chamber 12A and outflow chamber 12B. Pressure sensors 26A and 26B transmit a signal corresponding to a measured pressure to bypass valve controller 20E.

A control system which, in this example includes a computer 28 and a bypass valve controller 20E, computes a target variable value based on the signals received from pressure sensors 26A and 26B. In some embodiments, the target variable value is the peak differential pressure across valve V. In some embodiments, the target variable is the high pressure time. In some embodiments, the target variable is the cycle passing rate. The control system controls bypass valve 22, for example, according to any of the example control schemes described above or variations of those.

The control system includes a user interface that allows a user to look up information regarding the performance of the valve under test and the testing apparatus. The user interface may provide controls (for example, via computer 28) that allow a user to set parameters such as control limits, increments for controlling bypass valve 22, cycle rate, parameters that define the motion of piston 20A, and so on.

In some embodiments, an upper or lower control limit may be specified as a percentage in excess of a preset minimum value (e.g., a value prescribed by a testing standard). By way of example, ISO-5840 stipulates that prosthetic valves must be subjected to a peak differential pressure of at least 100 mmHg and that the peak differential pressure across the prosthetic valve must be maintained for at least 5% of the time per cycle (the high pressure time).

In some embodiments, a control limit may be inputted by specifying a percentage to cause the control limit to be set to the sum of 100% plus the specified percentage of a predetermined value such as a minimum value prescribed by an applicable standard. For example, if a user chooses the target variable to be the peak differential pressure across valve V, and ISO-5840 prescribes the peak differential pressure across the valve V to be at least 100 mmHg, then a user could set a lower control limit and/or an upper control limit for peak differential pressure by specifying a percentage value. For example, a user might input 2% for the lower control limit and 15% for the upper control limit. This would correspond to a lower control limit of 102 mmHg (i.e., 2% higher than 100 mmHg), and an upper control limit of 115 mmHg (i.e., 15% higher than 100 mmHg).

By way of further example ISO-5840 prescribes the minimum high pressure time to be 5% (i.e., in 5% of the period of a cycle a differential pressure of at least 100 mmHg must be maintained). A user could set the lower control limit for high pressure time by specifying 2%, for example, and the upper control limit for high pressure time by specifying 15%. This would translate to a lower control limit for the high pressure time of 5.1% (i.e., 2% higher than 5%), and an upper control limit of 5.75% (i.e., 15% higher than 5%).

At least one advantage of inputting the lower control limit and the upper control limit as percentages in excess of a minimum prescribed by a standard is that it simplifies selection of control limits that result in testing that satisfies requirements of applicable standards.

Bypass valve 22 can be controlled by the control system using any of the example control schemes described herein, for example.

Valve actuator 84 is mechanically coupled to bypass valve 22. Bypass valve controller 20E controls valve actuator 84 to set the opening of bypass valve 22 as required. In some embodiments, valve actuator 84 comprises a rotary actuator such as a servomotor or a stepper motor or a linear actuator.

In some embodiments, encoder 85 measures the degree to which bypass valve 22 is open and transmits a signal to bypass valve controller 20E corresponding to the degree to which bypass valve 22 is open.

In some embodiments, apparatus 10 comprises two or more bypass valves (e.g., 22 and 22′). Second bypass valve 22′ may have an opening that normally remains fixed in use. Second bypass valve 22′ may be adjusted in the event that bypass valve 22 is unable to keep the target variable below the upper control limit when completely open. In this case second bypass valve 22′ may be opened sufficiently so that the combined effect of bypass valves 22 and 22′ allows the target variable to be maintained within the corresponding range by controlling the degree of opening of bypass valve 22.

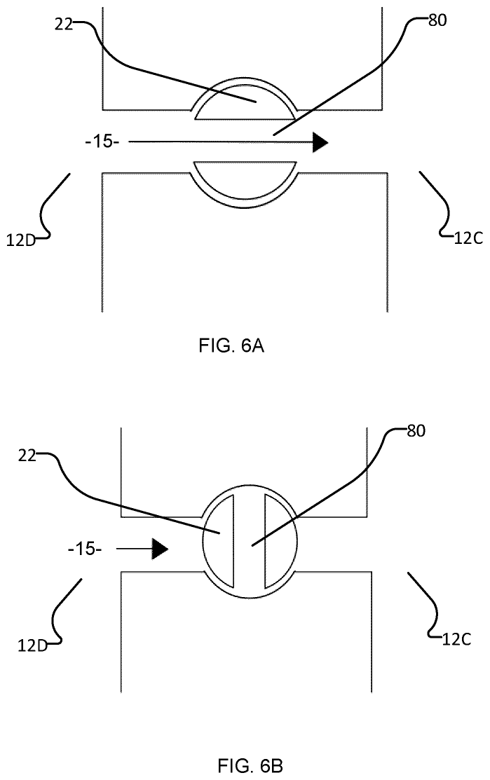

FIG. 6A shows an example bypass valve 22 in an open position according to an example embodiment. In the example embodiment shown in FIG. 6A, bypass valve 22 is a plug valve. Bypass valve 22 comprises a plug that is rotatable about an axis. An aperture 80 extends transversely through the plug such that fluid 15 can flow through bypass valve 22 between inflow path 12C and outflow path 12D when bypass valve 22 is in an open position. The cross sectional area of aperture 80 that is exposed to pass fluid 15 may be varied by rotating the plug.

FIG. 6B shows the bypass valve of FIG. 6A in a closed position so that no fluid 15 can flow through aperture 80 between inflow path 12C and outflow path 12D.

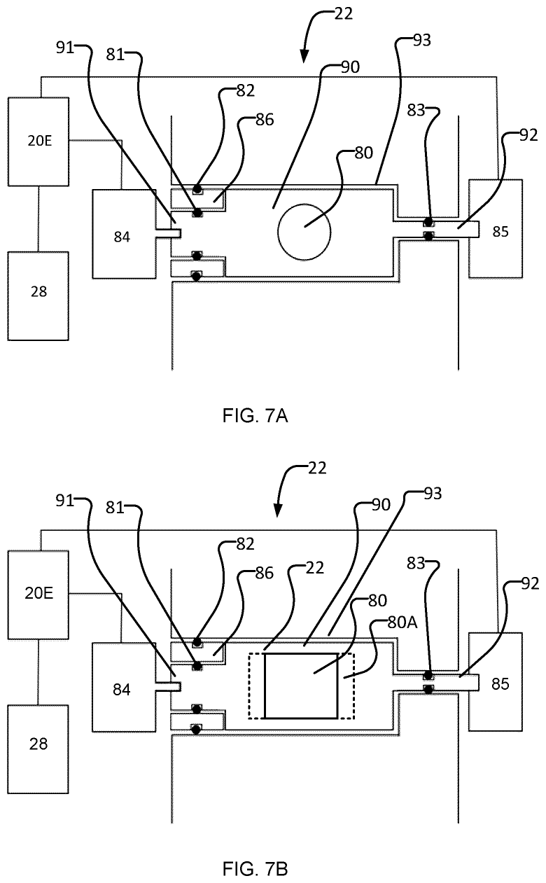

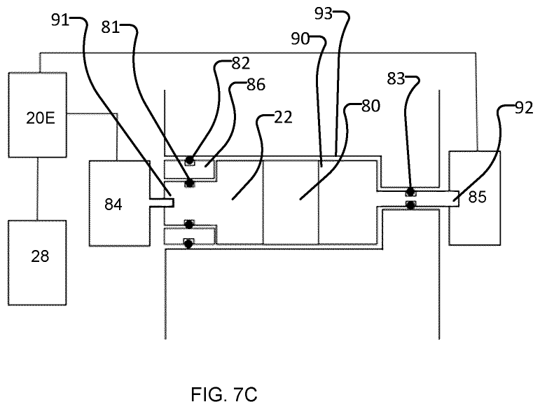

FIG. 7A is a schematic illustration of a bypass valve 22 according to an example embodiment. In the example embodiment shown in FIG. 7A, bypass valve 22 is in an open position such that fluid 15 (not pictured in FIG. 7A) can flow between inflow path 12C and outflow path 12D through aperture 80.

In some embodiments, bypass valve 22 is a cylindrical plug valve that has a rotationally symmetrical main plug body 90 that is supported to rotate in a valve body 93. Plug body 90 has a first axial end 91 and a second axial end 92. Main body 90 has a larger diameter than first axial end 91 and second axial end 92. Axial ends 91 and/or 92 may be tapered.

Use of a plug valve for bypass valve 22 is advantageous because plug valves have a relatively simple design and are easy to seal. Furthermore, the force required to open and close a plug valve does not increase significantly when a high pressure drop is present across the valve, unlike other types of valves (such as a butterfly valve). This is advantageous as the uniformly low force required to open and close the valve means that a less powerful valve actuator 84 can be used.

In some embodiments valve body 93 is formed integrally with a housing of apparatus as described herein.

Bypass valve 22 is mechanically coupled to valve actuator 84. Although the example embodiment of FIG. 7A shows valve actuator 84 coupled to first axial end 91 of bypass valve 22, in some embodiments valve actuator 84 is coupled to second axial end 92 of bypass valve 22.

In some embodiments, first axial end 91 is received in a bore of a collar 86. Seals 81, 82 and 83 prevent fluid 15 (not pictured in FIG. 7A) from escaping apparatus 10. This construction can help to reduce the force required to rotate plug body 90. First and second axial ends 91 and 92 (and seals 81 and 83) can both be significantly smaller in diameter than the rest of plug body 90. This reduces the frictional force by seals 81 and 83 which resist rotation of plug body 90. This results in less frictional force for valve actuator 84 to overcome. Less frictional force for valve actuator 84 to overcome means that valve actuator 84 can be less powerful, which is advantageous for at least the reason that a less powerful actuator may cost less and/or require less power to operate than a more powerful actuator.

In some embodiments seals 81, 82 and 83 are O-rings made of a silicone material with a hardness of about 70 (e.g., 60 to 80) on the Shore A Hardness Scale. O-rings of a sufficiently high hardness further reduce the friction that valve actuator 84 must overcome. The O-rings may be lubricated to further reduce friction.

FIG. 7A shows bypass valve 22 with an aperture 80 that is circular in cross section according to an example embodiment. In some embodiments, aperture 80 has a rectangular cross section. FIG. 7B shows an aperture 80 with a rectangular cross section according to an example embodiment. A rectangular aperture in a plug valve used as a bypass valve 22 has the advantage of providing a relatively linear relationship between the degree to which bypass valve 22 is open and the amount of aperture 80 open for fluid 15 to flow through. A rectangular cross section is also advantageous in that the total cross sectional area of aperture 80 can be changed by increasing or decreasing the length of aperture 80 (e.g., by replacing plug body 90 with a different plug body 90 that has an aperture 80 that has a different size in the axial direction along bypass valve 22, e.g., alternative aperture 80A).

An advantage of being able to change the cross sectional area of an aperture 80 is that different cross sectional areas may be more appropriate for testing valves that have different characteristics. The cross sectional area of aperture 80 may be selected depending on the valve V being tested. For example, if a small valve V intended for young children is being tested in apparatus 10, bypass valve 22 may be configured to have an aperture 80 that has a small cross sectional area for finer control. By contrast, if a larger valve V intended for an adult is being tested, bypass valve 22 may be configured to have an aperture 80 that has a larger cross sectional area. With a rectangular aperture 80, the total cross sectional area of aperture 80 can be easily modified by increasing the axial length of aperture 80 without changing the diameter of bypass valve 22.

In some embodiments, bypass valve 22 is comprised of plastic, for example, acetal resin.

In some embodiments, encoder 85 is coupled to either first axial end 91 of bypass valve 22, or second axial end 92 of bypass valve 22. Encoder 85 measures the degree to which bypass valve 22 is open. In embodiments where bypass valve 22 is a plug valve, this is done by measuring the degree of rotation of bypass valve 22.

In the example embodiment shown in FIG. 7A, encoder 85 relays a signal to bypass valve controller 20E corresponding to the degree to which bypass valve 22 is open. Bypass valve controller 20E may use the information regarding the degree to which bypass valve 22 is open to control a value of a target variable more precisely.

FIG. 7C shows bypass valve 22 in a fully closed position. In FIG. 7C, aperture 80 is oriented such that no fluid 15 can flow through aperture 80 between inflow path 12C and outflow path 12D.

FIG. 1 shows outflow compliance elements 24B in outflow chamber 12B and inflow compliance elements 24A in inflow chamber 12A. Inflow compliance elements 24A are designed to be compressed as a result of pressure increases and to expand as a result of pressure decreases in inflow chamber 12A. This reduces the magnitude and sharpness of the pressure increase in the first phase and of the pressure decrease in the second phase in inflow chamber 12A. Similarly, outflow compliance elements 24B are designed to reduce the magnitude and sharpness of the pressure wave in outflow chamber 12B.

In the illustrated embodiment, apparatus 10 includes pressure sensors 26A and 26B which respectively sense pressures in inflow chamber 12A and outflow chamber 12B. By way of non-limiting example, pressure sensors 26A and 26B may comprise Utah Medical model 6069 pressure transducers.

Output signals of pressure sensors 26A and 26B may be applied to control operation of apparatus 10 and/or to monitor the testing of valve V. For example, the motion of piston 20A (or other reciprocating member for driving fluid 15) and/or the operation of bypass valve 22 may be controlled at least in part based on the output signals of one or both of pressure sensors 26A and 26B. For example, the output signals of pressure sensors 26A and 26B may be logged and/or processed to yield other values such as differential pressure across valve V.

In some embodiments, outputs of pressure sensors 26A and 26B are connected to a data acquisition interface 27 as shown for example in FIG. 1. Interface 27 may, for example condition and/or digitize output signals from pressure sensors 26A and 26B. In some embodiments the output signals are delivered to a computer 28 for processing as shown for example in FIG. 1. Computer 28 may control overall operation of apparatus 10 including motion controller 20C.

As shown in FIG. 1, fluid 15 enters or exits outflow chamber 12B from a side of outflow chamber 12B, leaving end cap 17B un-obstructed for viewing valve V from the top of outflow chamber 12B. Similarly, fluid 15 enters or exits inflow chamber 12A from a side of inflow chamber 12A, leaving end cap 17A un-obstructed for viewing valve V from the bottom of inflow chamber 12A.

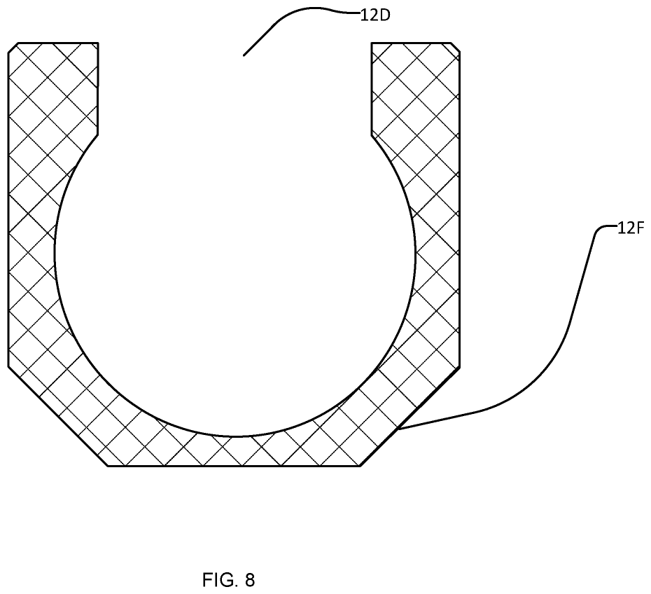

FIG. 8 is a cross sectional view of a housing of an example outflow chamber that may be provided in a valve testing apparatus. For example, a housing having the form illustrated in FIG. 8 may be used in a valve testing apparatus of the type shown in FIG. 1. The cross section of FIG. 8 is in a plane that passes through an opening at which outflow path 12D connects to outflow chamber 12B. The wall of the outflow chamber may be made with translucent material such that valve V is visible from outside apparatus 10. The outer surface of the wall of the outflow chamber may have multiple flat areas 12F through which valve V inside of the outflow chamber 12B may be viewed. Flat areas 12F reduce the distortion of views of valve V compared to a curved area. Cameras may be mounted to view valve V through the wall of outflow chamber 12B.

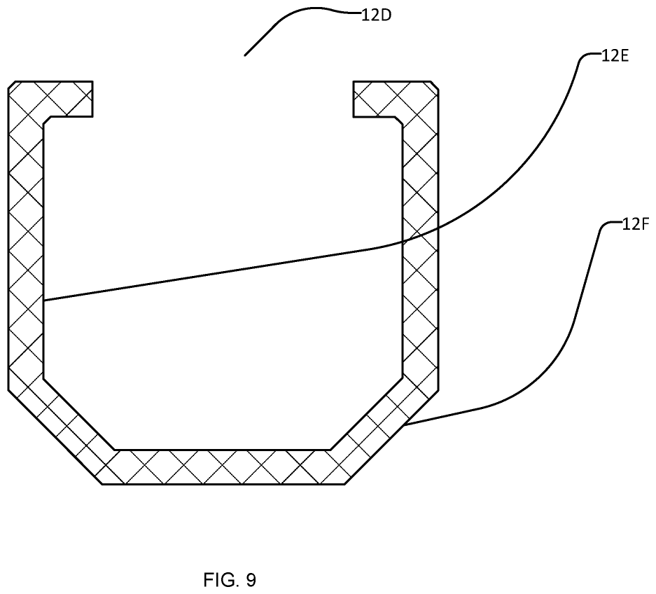

FIG. 9 is a cross sectional view of another example housing of an outflow chamber that may be provided in a valve testing apparatus. For example, a housing having the form illustrated in FIG. 9 may be used in a valve testing apparatus of the type shown in FIG. 1. The cross section of FIG. 9 is in a plane that passes through an opening at which outflow path 12D connects to outflow chamber 12B. The wall of the outflow chamber may be made with translucent material such that valve V is visible from outside apparatus 10. The outer surface of the wall of the outflow chamber may have flat areas 12F through which a valve V under test may be viewed. The inner surface of the wall of the outflow chamber may have flat areas 12E through which valve V may be viewed. Flat areas 12F and 12E reduce the distortion of views of valve V compared to a curved area.

Apparatus 10 may also include one or more cameras (not shown) that image valve V as it is being tested. Different cameras may be provided to image valve V from different sides and/or different angles. Acquired images may be useful in detecting and/or diagnosing problems that may occur with the operation of valve V during testing. Cameras may take snapshots of valve V. The snapshots may be triggered at specific points in the cycle of operation of apparatus 10. Cameras may take videos of valve V as it is tested. In some embodiments the videos are synchronized with data acquired from pressure sensors 26A, 26B and/or other sensors. Fluid 15 may be optically clear to facilitate the acquisition of high quality images of valve V. The cameras could be mounted on mounts at the ends of the test chamber, and could also be mounted on mounts on the sides of the test chamber (except for the side of the test chamber containing the piston).

In some embodiments pressure sensors 26A and 26B (as shown in FIG. 1, for example) each output a raw output signal. The output signals may, for example, each have a voltage indicative of the sensed pressure of fluid 15. The output signals are carried to data acquisition interface 27, for example by shielded cables.

In some embodiments data acquisition interface 27 conditions the output signals for example by amplifying the output signals and/or filtering the output signals. In an example implementation, an output signal from each of pressure sensors 26A and 26B is amplified to a range of +/−10 Volts and the output signals are low pass filtered. For example, low pass filters may be applied to remove signal noise at frequencies of about 7000 Hz and above. In some embodiments additional low pass filtering is applied to each output signal. A cutoff frequency of the additional filtering may be variable. For example, a cutoff frequency in the range of about 30 Hz to about 1 kHz may be set under software control. The cutoff frequency for the additional filtering may be continuously variable or selectable from a plurality of discrete cutoff frequencies (for example, cut off frequencies of 30 Hz, 100 Hz, 300 Hz, and 1000 Hz). The conditioned output signals are digitized and then provided to computer 28 for further processing. Filtering may be performed in the analog domain and/or the digital domain.

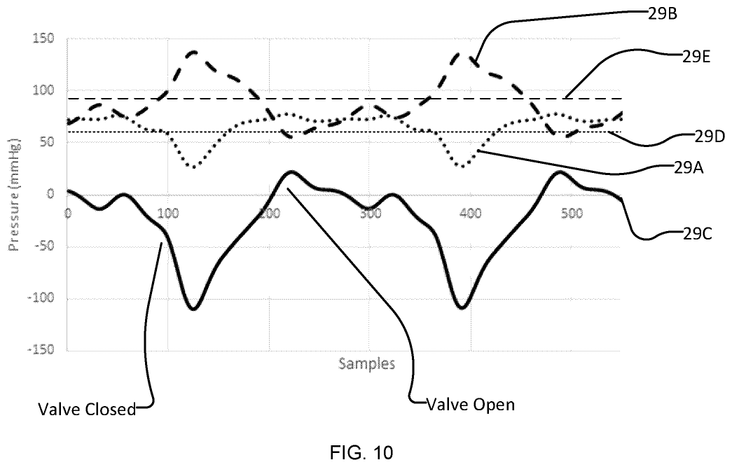

FIG. 10 is an example graph in which curve 29B represents pressure in outflow chamber 12B (as detected by pressure sensor 26B) as a function of time, curve 29A represents pressure in inflow chamber 12A (as detected by pressure sensor 26A) as a function of time, and curve 29C represents the differential pressure across valve V between inflow chamber 12A and outflow chamber 12B as a function of time. Curve 29C shows the difference between the inflow chamber pressure and the outflow chamber pressure (i.e. the negative of the differential pressure which is the difference between the outflow chamber pressure and the inflow chamber pressure). This has been done for the sake of clarity in FIG. 10.

The pressure values of curve 29A and curve 29B may be processed to obtain mean pressure values shown in curve 29D and curve 29E, respectively. The mean pressure values of 29D and/or 29E may be used to verify proper operation of apparatus 10 (e.g., by computer 28). A significant change (a value ranging between 3 mmHg and 5 mmHg, for example) in mean pressure 29D over time may indicate that pressure sensor 26A is drifting and requires recalibration. Similarly, a significant change in mean pressure 29E over time may indicate that pressure sensor 26B is drifting and requires recalibration. In addition, a sudden significant change in both mean pressure 29D and mean pressure 29E may indicate fluid leakage, valve failure, or other unexpected changes to the testing conditions. Such changes may be automatically detected by processing in computer 28. The changes may trigger a warning indication to be provided to an operator and/or stop operation of the system.

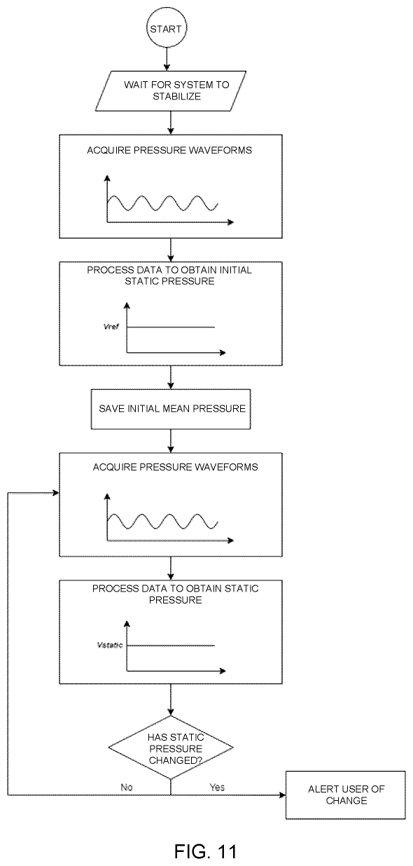

FIG. 11 is a flow chart for an automatic pressure verification system. The fluid pressure in test chamber 12 is measured at one or more locations (e.g., by one or both of pressure sensors 26A, 26B and/or one or more other pressure sensors).

An output signal from each of one or more sensors is processed to determine the mean pressure at the location of the sensor. The mean pressure observed at each sensor may be detected by processing the signals from different pressure sensors 26 independently during system operation. The data may be processed as raw voltage values or pressure values.

As shown in FIG. 11 an initial mean pressure reference may be determined at or near the start of a test. As the test progresses the software compares the mean pressure to the initial mean pressure reference. If the difference is considered unacceptable by a pre-determined metric (e.g., the difference exceeds a threshold amount) then an action may be triggered (e.g., triggering an alarm, sending a message, logging an event, stopping the system, etc.) FIG. 11 illustrates this program flow.

The software may continuously process sampled pressure data at a sampling frequency that accommodates for the operational drive frequency of the apparatus. Individual periods of the resulting periodic pressure waveform may be identified by using sync signals (e.g., signals generated by motion controller 20C-typically short pulses at starts of reciprocation cycles). The data may be processed over one or more periods of the pressure signal to obtain the mean pressure.

Methods for determining the mean pressure include:
Applying an averaging algorithm to obtain a mean signal value. In some embodiments the averaging algorithm is iterative and uses a fixed or running window. For example, the processing may comprise determining a DC component of the pressure signal being processed, for example, by integrating the pressure signal over one or more periods of the pressure signal and dividing the integration value by the length of the integrated portion of the pressure signal;
Applying a finite digital filter to the pressure sensor signal. The digital filter may have a cut off frequency between 0.1-0.01 Hz, in order to obtain the mean signal for each period; and/or
Applying a Fast Fourier Transform to the pressure sensor signal. The mean pressure corresponds to the value at 0 Hz in the transformed signal.

In some embodiments, mean pressure is calculated for individual periods of the pressure waveform (e.g., every period, every nth period, one period every so often—e.g., once per second, once per minute etc.). In some embodiments mean pressure is calculated over windows that are many times as long as one period. In some embodiments calculated mean pressures for individual periods of the pressure waveform or for individual windows that may be longer than one period are saved (e.g., for 5-50 periods of the pressure waveform). The saved values may be combined to decrease variability and produce a more accurate mean pressure reading (e.g., by averaging, averaging after discarding outliers, etc.). The window size used for individual determinations of mean pressure may be pre-determined.

The reference mean pressure may be obtained as described above at an initial stage of the test after the system has stabilized in operation. In addition, or in the alternative, the reference mean pressure may be measured at any point in the testing process. The reference mean pressure may be stored and compared to subsequent mean pressure values determined during testing.

Some embodiments include performing statistical analyses of the mean pressures measured during a test. For example, mean pressures computed at different times may be processed to determine statistics such as the average, median, variance, etc. of the mean pressure.

In some embodiments, throughout testing, the software regularly (e.g., every fixed number of periods) obtains the system mean pressure. The system mean pressure and/or an average of a specified number of most recent values for system mean pressure are compared to the initial mean pressure reference. If the mean pressure starts to deviate from the reference mean pressure consistently and significantly, the system will alert the user of this change. The criteria used to warrant this alert may be determined based on user-determined variance values. For example, if the mean pressure falls outside of 10% variance of the mean pressure reference for a determined number of consecutive cycles, a warning may be issued.

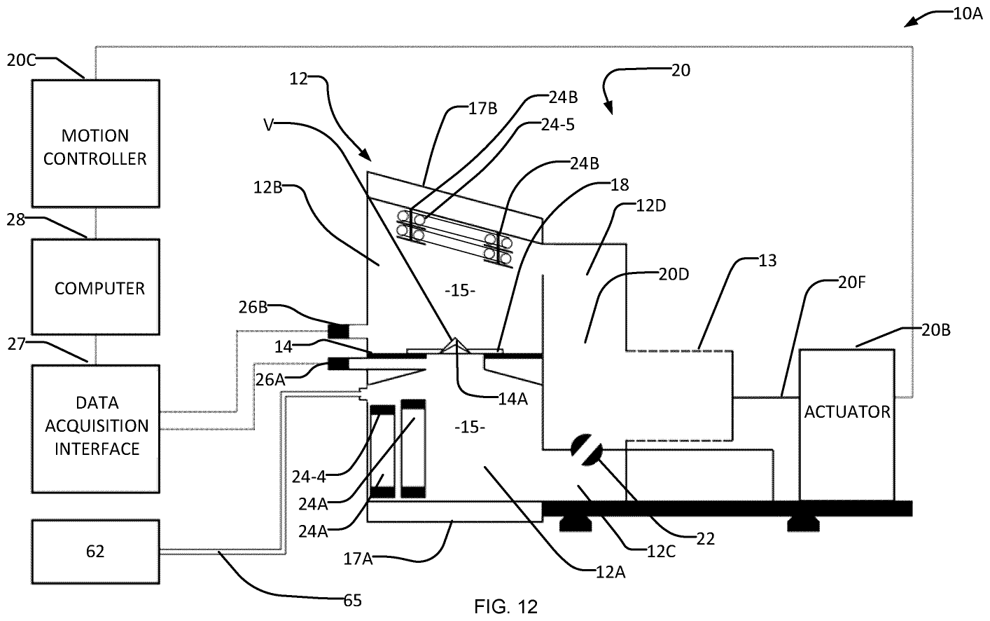

FIG. 12 shows an apparatus 10A which is similar to apparatus 10 with the main exception that piston 20A is replaced with a bellows 13. The same reference numbers are used to indicate other elements present in both apparatus 10 and 10A.

The expansion and contraction of bellows 13 as shaft 20F reciprocates forward and backward generates pressure differentials across valve V and drives fluid 15 around the system.

As shaft 20F advances (moves to the left in FIG. 12) fluid 15 is pushed through outflow path 12D into outflow chamber 12B. This increases the pressure in outflow chamber 12B and creates a pressure differential across valve V between inflow chamber 12A and outflow chamber 12B, which causes valve V to close. As valve Vis closes, some fluid 15 will flow through valve V into inflow chamber 12A.

As shaft 20F continues to advance, pressure in outflow chamber 12B will rise to provide the required differential pressure across valve V. Under this rising differential pressure between inflow chamber 12A and outflow chamber 12B, bypass valve 22 allows fluid to flow from outflow chamber 12B into inflow chamber 12A to limit the peak differential pressure across valve V so that it does not exceed the required differential pressure by more than a set amount. The waveform of the pressure in outflow chamber 12B is also smoothed by outflow compliance elements 24B due to their compression under rising pressure and expansion under falling pressure in outflow chamber 12B.

As shaft 20F continues to advance, and more fluid 15 is delivered to inflow chamber 12A through bypass valve 22, the pressure in inflow chamber 12A will rise. Inflow compliance elements 24A may be designed to take all the fluid 15 driven into the inflow chamber from the outflow chamber (through compression of inflow compliance elements 24A under rising pressure) before shaft 20F is fully advanced. Inflow compliance elements 24A effectively reduce the magnitude of the pressure increase in inflow chamber 12A as well as smooth the pressure waveform.

When shaft 20F reaches the end of its stroke and starts to retract, the volume of outflow path 12D begins to increase. This increase in volume draws fluid 15 out of outflow chamber 12B and consequently reduces the pressure in outflow chamber 12B. When the pressure in outflow chamber 12B is reduced below the pressure in inflow chamber 12A the pressure differential across valve V will cause fluid 15 to flow through the valve from inflow chamber 12A to outflow chamber 12B, forcing valve V to open. As fluid 15 leaves inflow chamber 12A, inflow compliance elements 24A will expand. The cycle may then be repeated.

In apparatus as described herein it is beneficial to maintain a pressure in fluid 15 to be slightly higher than the ambient pressure of air surrounding the apparatus throughout the testing. Maintaining fluid 15 at a slight positive pressure tends to resist the ingress and accumulation of air inside the apparatus. By contrast, if the pressure inside apparatus 10 dips below the ambient pressure then air may be drawn into apparatus 10 where the air may accumulate and form bubbles. Bubbles of air in fluid 15 may interfere with obtaining good images of valve V and/or affect the testing of valve V.

Apparatus 10A of FIG. 12 as well as apparatus 10 of FIG. 1 advantageously facilitates maintaining the mean pressure of fluid 15 in inflow chamber 12A above, but only slightly above the ambient pressure outside of inflow chamber 12A throughout testing. As mentioned above, the peak differential pressure between inflow chamber 12A and outflow chamber 12B must reach a certain value during testing.

Apparatus 10A as well as apparatus 10 can achieve the desired differential pressure while keeping the mean pressure of fluid 15 relatively low primarily by increasing the pressure within outflow chamber 12B when valve V is closed during which time a high peak differential pressure across valve V is required.

In addition, inflow compliance elements 24A may have relatively large compressible volume (e.g., 50 ml for apparatus 10 of FIG. 1 or 240 ml for apparatus 10A of FIG. 12) to reduce the amplitude of pressure waveforms in inflow chamber 12A. Inflow compliance elements 24A may have even larger compressible volume than specified above, but the benefit of the extra volume beyond the specified level may diminish. This reduced amplitude of pressure waveforms in inflow chamber 12A helps to facilitate operating apparatus 10 at a low mean pressure while still keeping the whole pressure waveform in inflow chamber 12A above ambient pressure. The large compliance volume in inflow chamber 12A compresses as fluid enters and expands as fluid leaves inflow chamber 12A. Thus a relatively stable pressure near the mean pressure is maintained in inflow chamber 12A. Therefore, the mean pressure can be maintained at a level that is comparatively significantly lower than would otherwise be possible while still achieving suitable pressure differentials across valve V.

By contrast, if outflow chamber 12B has a high compliance (e.g., if introducing a unit of fluid volume into outflow chamber 12B results in relatively little increase in the pressure of outflow chamber 12B) then it may not be practical to achieve the required pressure differential by raising the pressure within outflow chamber 12B. In such cases it could be necessary to achieve the desired pressure differential by reducing pressure in inflow chamber 12A. To do this while keeping the mean pressure in inflow chamber 12A above ambient pressure all the time it would be necessary to maintain a relatively high mean pressure within inflow chamber 12A (e.g., a pressure that is at least slightly greater than the desired pressure differential).

Maintaining a high mean pressure in inflow chamber 12A will result in an even higher mean pressure in outflow chamber 12B, as the mean pressure in outflow chamber 12B is typically higher than that in inflow chamber 12A. Maintaining a high mean pressure in both inflow chamber 12A and outflow chamber 12B has disadvantages including requiring more force to drive bellows 13 or other mechanism for driving flow of fluid 15, higher possibility of leaks, greater rate of wear on flexible components, and requiring test chamber 12 to be constructed more robustly. Maintaining reduced mean pressure has the advantage of reducing outward fluid leakage and evaporation, reducing the wear rate on flexible system components, and reducing load on the drive actuator.

One aspect of the present invention provides a construction for compliance elements suitable for use in accelerated wear testing systems. Another aspect of the present invention provides accelerated wear testing systems that provide specific amounts of compliance in different parts of the systems. These aspects may be applied individually or in combination.

Compliance elements may take the form of tubes. The interior of the tubes may be filled with a compressible gas (e.g., air). The compressible gas may be at an initial pressure of 1 atm or another pressure that is slightly less or more than 1 atm. The material of the tubes is impermeable to the contained gas and the tubes are sealed so that the contained gas cannot escape into fluid 15.

In some embodiments, compliance elements have the form of thin walled hollow tubes that are closed at either end (e.g., by plugs or seals) or formed into closed loops. The material of the walls of the tubes may be a soft flexible material such as silicone. In some embodiments the material of the tubes has a hardness in the range of 30 to 40 on the Shore A scale. In some embodiments the tubes have a wall thickness in the range of 0.5 mm to 1.5 mm (e.g., approximately 1 mm). In some embodiments the tubes of compliance elements 24 have an external diameter or external diameters in the range of about 5 mm to about 30 mm or about 7 mm to about 9 mm. Some embodiments include compliance elements 24 comprising sections of tubing that are plugged at both ends and/or sections of tubing that form a closed loop with ends joined by a plug or connector.

Preferably compliance elements 24 are supported in test chamber 12 by supports which hold the compliance elements 24 in place but permit the compliance elements to be easily removed and replaced as desired. Compliance elements 24 may for example be received in grooves or channels in walls of test chamber 12. Other examples of supports include clips, guides, or other holding elements. Clips may, for example have hooks or other openings that are smaller than the compliance elements when uncompressed, but which can receive the compliance element when compressed.

The number, size, and locations of compliance elements 24 may be selected to achieve desired levels of compliance in inflow chamber 12A and outflow chamber 12B. In some embodiments a total internal sealed volume of compliance elements 24 in inflow chamber 12A is at least 15 ml (e.g., in the range of 15 ml to 50 ml) for apparatus like apparatus 10 of FIG. 1 or at least 80 ml (e.g., in the range of 80 ml to 240 ml) for apparatus like apparatus 10A of FIG. 12. Compliance elements 24B in outflow chamber 12B are optional and if present may, for example, have internal sealed volumes in the range of 0 ml to 50 ml.

It can be beneficial to provide at least some compliance elements 24 in close proximity to valve V. For example, in some embodiments, at least one compliance element 24 is located no farther than 100 mm from valve V (by the closest path through fluid 15).

Tubular compliance elements are advantageous compared to compliance elements that include flexible sheets clamped along their edges. With tubular compliance elements, forces are more evenly distributed along the surface of the material of the tubing. By contrast, sheets that are clamped along their edges are easily subjected to tears. Other advantages of tubular compliance elements as compared to other types of compliance elements are that tubular compliance elements may be easily secured by simple restrictive devices such as clip holders, ring holders, and wires; and may be easily installed, taken out, or adjusted in terms of number of tubular compliance elements and total compliance volume. In some embodiments the total number of compliance elements 24 is between 3 and 8 in inflow chamber 12A and between 2 and 6 in outflow chamber 12B.

The presence of compliance elements 24 helps to avoid or attenuate undesirable pressure oscillations or spikes that may otherwise occur as a result of valve V opening or closing and/or fluid flows into or out of testing chamber 12. The presence of compliance elements 24 allows the volume of fluid 15 contained in inflow chamber 12A or outflow chamber 12B to change to avoid excessive pressure changes.

Compliance elements 24 may be arranged in many ways, for example, circular, following an arc, following a helix or spiral, and following a straight path.

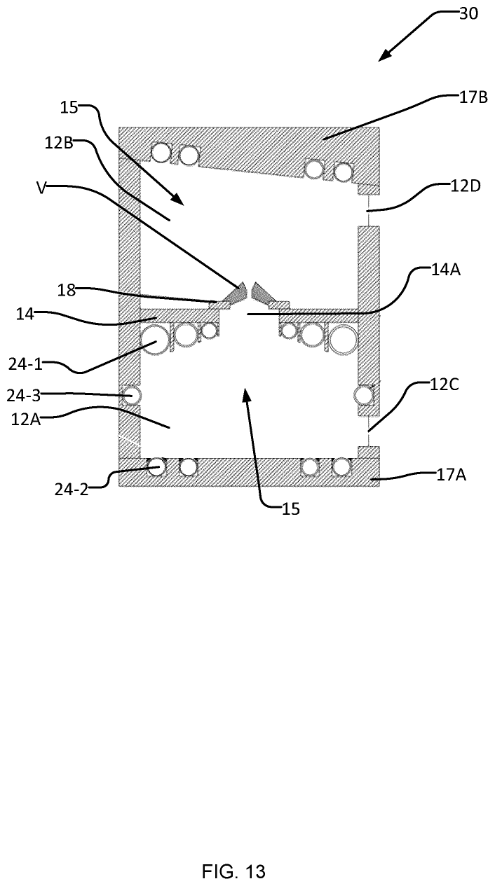

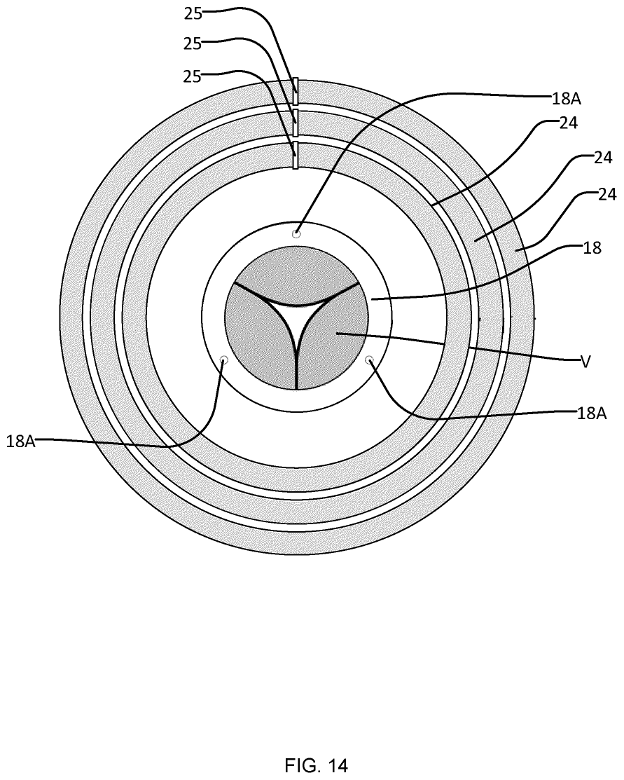

FIG. 13 is a cross sectional view of a test chamber 30 according to an example embodiment. Test chamber 30 may, for example, be applied in apparatus 10 or 10A. FIG. 1, FIG. 2 and FIG. 3 illustrate various example locations for compliance elements 24. Test chamber 30 includes elastomeric compliance elements 24 in several example locations. Systems 10 and 10A include compliance elements 24 in other example locations. Example locations for compliance elements 24 include:
on partition 14 in inflow chamber 12A (for example extending around mount 18, for example in one or more closed loops or circles concentric with mount 18 (as indicated by 24-1, as in FIG. 13);
on an end wall of testing chamber 12 in inflow chamber 12A (for example in a groove formed in the end wall or between projections from the end wall, for example, the compliance element may extend in a loop which may be circular (as indicated by 24-2, as in FIG. 13);
on a side wall of testing chamber 12 in inflow chamber 12A (for example, compliance elements may extend circumferentially around the side wall as indicated by 24-3 or in an axial direction as indicated by 24-3, as in FIG. 13);
on an end wall of testing chamber 12 in outflow chamber 12B (as indicated by 24-4, as in FIG. 12); and/or
in a stack of two or more layers of tubular compliance elements (as indicated for example by 24-5, as in FIG. 12).

Typically, sufficient compliance can be achieved with compliance elements 24 in only some of these locations. Many single embodiments will have compliance elements 24 in a smaller number of locations.

Compliance elements 24 may, for example each have an internal volume of 5 ml-10 ml for circular rings or 20 ml-60 ml for straight tubing.

In some embodiments the total interior volume of compliance elements 24 in outflow chamber 12B is in the range of 0 to 30 cubic centimeters. In some embodiments the interior volume of compliance elements 24 in inflow chamber 12A is in the range of 30 to 50 cubic centimeters.

As mentioned above, compliance elements 24 may be arranged in concentric closed loops (e.g., concentric circles). For example, in some embodiments two or more compliance elements 24 are arranged in concentric loops centered on aperture 14A on the side of partition 14 facing inflow chamber 12A. FIG. 14 illustrates an example of this construction. In FIG. 14, the concentric loops are formed by sections of tubing having their ends joined by couplers 25.

In some embodiments the compliance elements include tubes having different diameters. For example, smaller diameter tubes may, be used for compliance elements that follow more tightly curved (smaller radius) paths and larger diameter compliance tubes may be used for compliance elements that follow less tightly curved (larger radius) paths. Other benefits of using compliance elements of two or more different diameters of tubing can include more options for providing a desired degree of compliance volume within a particular test chamber and creating a sloped surface which tends to reduce the likelihood that any air bubbles in fluid 15 could stick to and stay trapped on the surface. An embodiment with several tubes of the same diameter would be suitable as well. In such a case, the bottom side of partition 14 may have a slanted surface to allow air bubbles to move up towards valve V. Tubing of 1 mm wall thickness and durometer 35A provides a thin enough membrane for an adequate compliance in the illustrated embodiment, however different tubing may also be sufficient.

In some embodiments, compliance is provided by plural compliance elements 24 that have the same tubular diameter or ring diameter.

The compliance of inflow chamber 12A and outflow chamber 12B may be individually adjusted by adding or removing compliance elements 24. This may be done to tailor the apparatus for a particular testing protocol. For example, the apparatus may be adjusted to increase a pressure applied on a valve V when closed by removing one or more compliance elements 24 from outflow chamber 12B and/or replacing one or more compliance elements 24 in outflow chamber 12B with other compliance elements 24 that have lower compliance. Such adjustments may be made, for example to configure apparatus as described herein to apply very high pressure differentials (e.g. 315 mmHg), as may be desired for failure mode tests. Adjustment may also be made, for example to configure the same apparatus for performing longevity tests at lower pressure differentials such as 100 mmHg or 120 mmHg. These adjustments to compliance may be combined with changing the resistance to flow of bypass valve 22. Setting bypass valve 22 to provide more resistance to flow increases maximum pressures in outflow chamber 12B whereas setting bypass valve 22 to provide less resistance to flow decreases maximum pressures in outflow chamber 12B.

As mentioned above, it can be beneficial to operate systems as described herein under conditions such that the mean pressure within inflow chamber 12A is very slightly higher than ambient pressure. A system as described herein may include a pressurization system that maintains the mean pressure in inflow chamber 12A within desired limits.

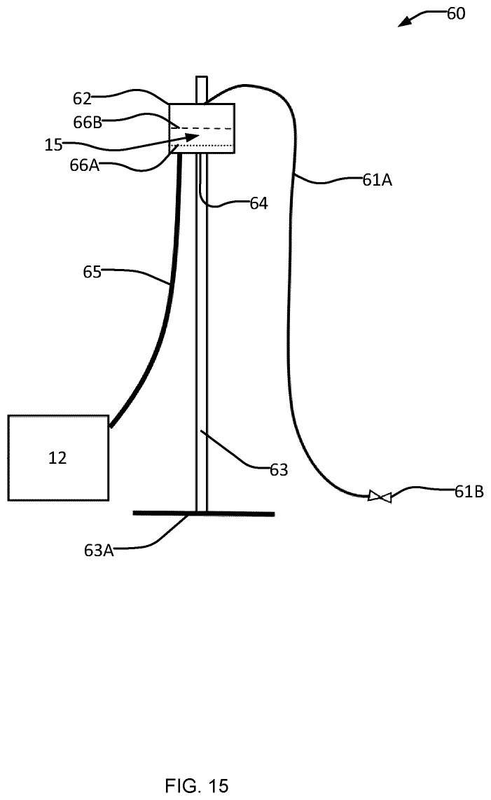

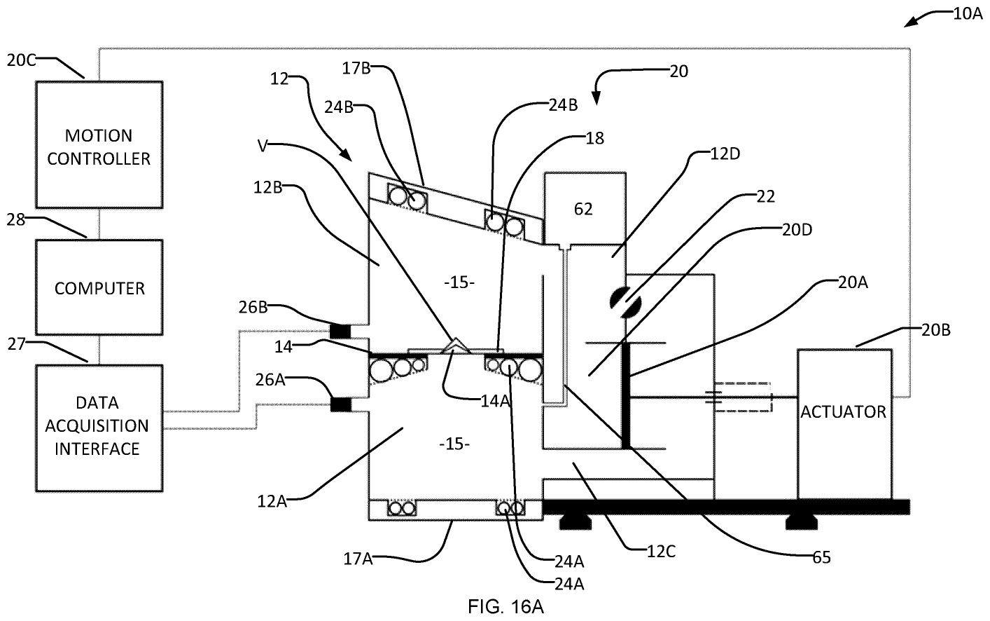

FIG. 15 shows an example pressure reference system 60 that is operable to maintain a desired mean pressure within inflow chamber 12A. Pressure reference system 60 also provides a reservoir of appropriate test fluid 15 which may automatically replenish any fluid 15 that may be lost from test chamber 12 (e.g., by evaporation) during a test.

Pressure reference system 60 comprises a reservoir 62 dimensioned to contain a defined volume of fluid 15. Reservoir 62 is mounted at a set elevation above valve V in a test chamber 12. In the illustrated embodiment, reservoir 62 is attached to a support pole 63 by an adjustable coupling 64 which is movable along support pole 63 to place reservoir 62 at a set height. Support pole 63 may be attached to a base 63A which can be set on a flat surface or may be attached to another convenient structure.

Reservoir 62 is fluidly coupled to inflow chamber 12A of a test chamber 12 by a conduit 65. Conduit 65 may, for example, be flexible tubing. Fluid 15 in reservoir 62 is in fluid communication with fluid 15 in inflow chamber 12A of test chamber 12 via a conduit 65. The fluid level in reservoir 62 may be at an elevation higher than valve V inside test chamber 12 to cause mean pressure in inflow chamber 12A to be elevated. The mean pressure in test chamber 12 may be increased by 1 mmHg by raising the fluid level in reservoir 62 by about 1.36 cm (in the case where fluid 15 has about the same density as water). For example, to maintain a mean pressure of about 50 mmHg in inflow chamber 12A, the level of fluid 15 in reservoir 62 may be maintained at about 68 cm above valve V.

Reservoir 62 may be made of translucent material or have a translucent window so that the fluid level inside reservoir 62 is visible.

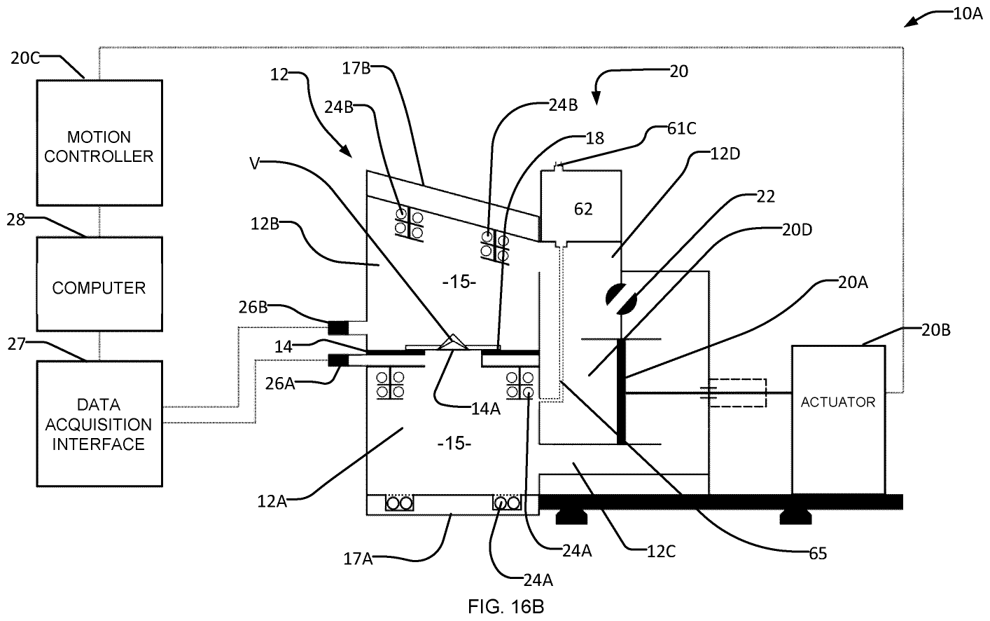

FIG. 16A shows another example pressure reference system 60 that has reservoir 62 placed directly on top of test chamber 12. In the illustrated embodiment, reservoir 62 is fluidly coupled to inflow chamber 12A by conduit 65. Conduit 65 may, for example, be an internal tubing, conduit or channel passing through test chamber 12 to fluidly couple reservoir 62 to inflow chamber 12A.

FIG. 16B shows another example pressure reference system 60 that has reservoir 62 placed directly on top of test chamber 12. In the example embodiment shown in FIG. 16B, reservoir 62 is vented by ventilation port 61C to equalize the pressure inside reservoir 62 with the ambient pressure. Inflow compliance elements 24A and outflow compliance elements 24B are mounted in stacks of plural inflow compliance elements 24A and plural outflow compliance elements 24B.

Connecting liquid reservoir 62 directly to inflow chamber 12A is effective to maintain a positive pressure in apparatus 10. Where reservoir 62 is directly connected to inflow chamber 12A close to valve V then sufficient pressurization of apparatus 10 may be achieved when the level of fluid 15 in reservoir 62 is as little as about 100 mm above valve V. The positive pressure imposed by reservoir 62 at the outlet in inflow chamber 12A may be as little as 5 to 10 mmHg. Since the mean pressure in outflow chamber 12B during test is always higher than the mean pressure in inflow chamber 12A, keeping the mean pressure in inflow chamber 12A above zero will keep the mean pressure in outflow chamber 12B above zero. This configuration therefore has significant advantages over test machines in which a pressure source is connected to the outflow chamber, including the advantage that the reservoir 62 may be much lower.

Reservoir 62 may be fluidly connected to fluid 15 in inflow chamber 12A through conduit 65 with one end point in inflow chamber 12A. Ideally the end point in inflow chamber 12A is close to valve V (e.g., preferably no further than 40 mm away from valve V). In some embodiments, conduit 65 has a small diameter. Without wishing to be bound by theory, it is posited that a small diameter conduit 65 allows a positive pressure to be maintained in test chamber 12 while also limiting the fast pressure variations imposed by the reciprocating member on the fluid in reservoir 62. This is because a small diameter conduit 65 acts similarly in function to an inductor in an electrical circuit. A small diameter conduit 65 opposes short-term changes in pressure on the fluid in reservoir 62 while also permitting a positive pressure to be maintained in test chamber 12. In some embodiments, conduit 65 has a diameter small enough to limit pressure variations on the fluid in reservoir 62 while also having a diameter large enough to facilitate cleaning the interior of conduit 65. In some embodiments, conduit 65 has a diameter of 5 mm.

The vertical distance between the fluid level in reservoir 62 and valve V may for example be in the range of about 60 mm to about 1.5 m.

Mean pressure in inflow chamber 12A may be kept constant during a test by maintaining the level of fluid 15 in reservoir 62 within a specified range so that the mean pressure in inflow chamber 12A is constant to within a specified accuracy throughout a test. For example, the level of fluid 15 inside reservoir 62 above valve V may be maintained to remain within a range of ±6.5 mm to keep variation of the mean pressure in inflow chamber 12A to be within ±1 mmHg. For example, if the level of fluid in reservoir 62 is 100 mm±6.5 mm above valve V during a test then the mean pressure in inflow chamber 12A near valve V would be in the range of 7±1 mmHg for the duration of the test.

Reservoir 62 may be marked with indicia 66 showing a desired surface level for fluid 15 in reservoir 62. In the illustrated example embodiment shown in FIG. 15, indicia 66 includes indicia 66A showing a minimum level for fluid 15 and indicia 66B showing a maximum fluid level for fluid 15. Mean pressure in inflow chamber 12A may be maintained within a desired range by keeping the fluid level in reservoir 62 between indicia 66A and 66B. Preferably the level of fluid 15 in reservoir 62 is maintained at a level that is between indicia 66A and indicia 66B throughout a test.

The vertical height difference between indicia 66B and 66A may range between 10 mm and 70 mm. If the level of fluid 15 in reservoir 62 is maintained between indicia 66B and 66A, the static pressure generated by the reservoir shall have an accuracy ranging between about +/−0.4 mmHg and about 2.6 mmHg.

The vertical height difference between indicia 66B and 66A may, for example, be in the range of 13 mm to 40 mm. If the level of fluid 15 in reservoir 62 is maintained between indicia 66B and 66A, the static pressure generated by the reservoir shall have an accuracy ranging between about ±0.5 mmHg and about ±1.5 mmHg.

In addition to maintaining a desired mean pressure in inflow chamber 12A, any fluid that is depleted from test chamber 12 (e.g., by evaporation or leakage) may be automatically replenished with fluid 15 from reservoir 62. Reservoir 62 can also serve as a vent when it is desired to drain fluid 15 out of test chamber 12 as well as a way to introduce fluid 15 to fill reservoir 12 after reservoir 12 has been drained.

Reservoir 62 may be enclosed or have a lid to reduce the area of fluid 15 exposed to the external atmosphere, thereby reducing fluid loss by evaporation. If reservoir 62 is enclosed then the enclosed volume above the level of fluid 15 in reservoir 62 is vented to ensure that reservoir 62 is an open system with exposure to atmospheric pressure so that unintended pressure build up or pressure vacuums in the enclosed reservoir 62 will not alter pressure in test chamber 12.

In some embodiments reservoir 62 is enclosed and vented by way of a vent line 61A with an opening inside reservoir 62 at a location above the maximum fluid level. A control valve 61B may be provided. Control valve 61B is operable to open or restrict flow of fluid through vent line 61A. Control valve 61B may be adjusted to control the rate at which test chamber 12 is filled with fluid 15 from reservoir 62 or drained from test chamber 12. Control valve 61B may, for example, comprise a two-way valve, a tubing shut off clamp, or an adjustable flow restrictor. Reservoir 62 may include a small hole or opening above the maximum fluid level for equalizing pressure inside reservoir 62 with pressure outside of reservoir 62.

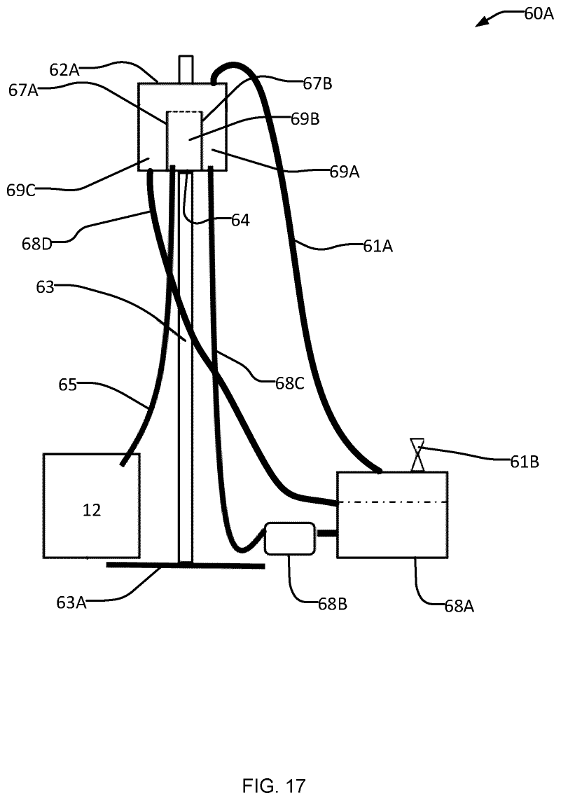

FIG. 17 shows another example pressure reference system 60A. Those elements of pressure reference system 60A that are identified by references also used to identify elements of pressure reference system 60 (in FIG. 15) have the same or similar functions as described with respect to pressure reference system 60.

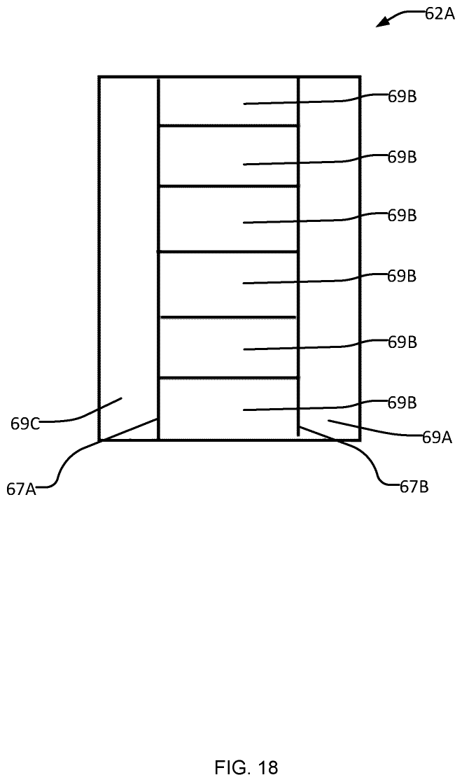

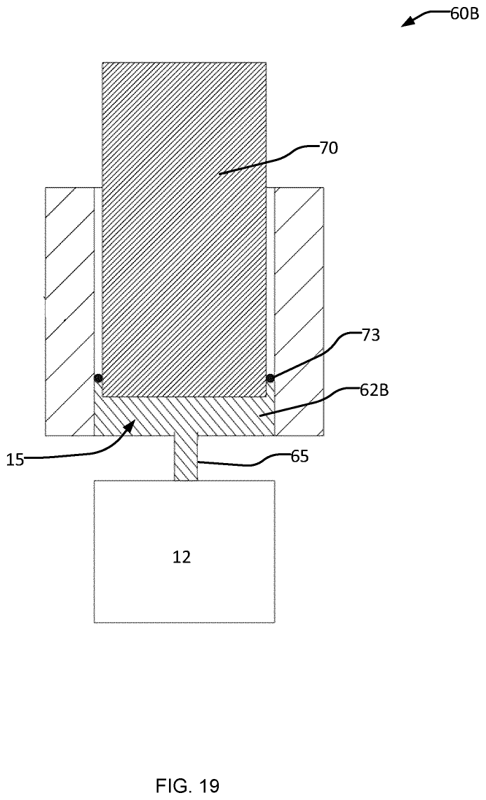

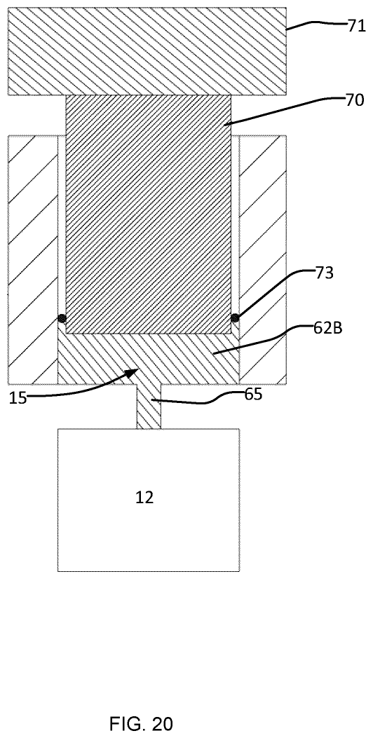

Pressure reference system 60A includes a reservoir 62A that contains fluid 15 having a surface at an elevation above test chamber 12 so as to maintain a desired static pressure within test chamber 12. FIG. 18 is a top view of an example construction for reservoir 62A. Pressure reference system 60A differs from pressure reference system 60 in that the level of the surface of fluid 15 in reservoir 62A is defined by a barrier 67A over which fluid 15 can spill and that pressure reference system 60A includes a return fluid tank 68A and a recirculating pump 68B connected to deliver fluid 15 into reservoir 62A.

Fluid 15 delivered into reservoir 62A fills reservoir 62A up to the level of barrier 67A. Excess fluid 15 spills over barrier 67A into an outgoing fluid cell 69C from where the fluid 15 is conveyed to return fluid tank 68A through line 68D.

In the illustrated embodiment, reservoir 62A includes three sections, an incoming fluid cell 69A into which fluid 15 is delivered by pump 68B, one or more test fluid cells 69B which are in fluid communication with one or more corresponding test chambers 12, and the outgoing fluid cell 69C. Incoming fluid cell 69A is separated from test fluid cells(s) 69B by a barrier 67B. When pump 68B is operating, fluid 15 is delivered into incoming fluid cell 69A which becomes filled with fluid 15. Fluid 15 then spills over barrier 67B into test fluid cell(s) 69B. When test fluid cells 69B are filled to the level of barrier 67A fluid 15 spills over barrier 67A into outgoing fluid cell 69C.

Some embodiments include plural test fluid cells 69B. Each of test fluid cells 69B may be in fluid communication with a corresponding test chamber 12 by a corresponding conduit 65. If a connected test chamber 12 consumes any fluid from a corresponding test fluid cell 69B then the test fluid chamber is replenished with fluid 15 delivered by pump 68B through line 68C.

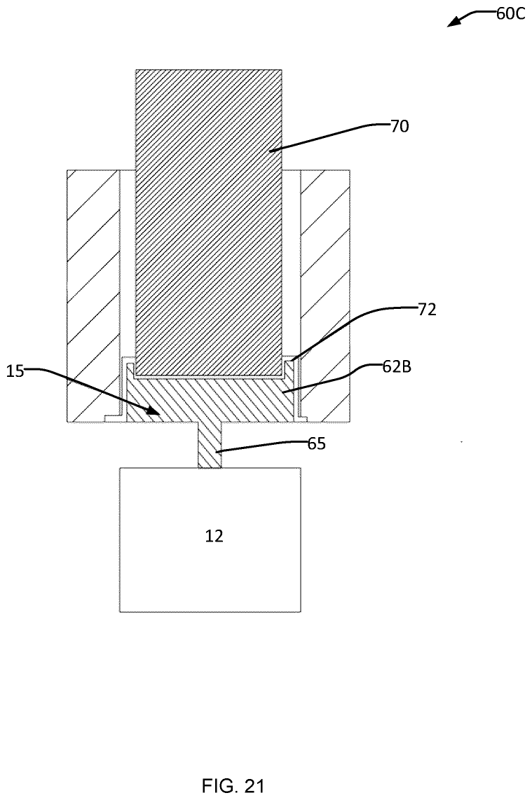

FIG. 19 is a schematic view showing another example pressure reference system 60B. System 60B applies the force of gravity operating on a weighted piston 70 to pressurize fluid 15 in a reservoir 62B. Weighted piston 70 is mounted to slide vertically and is sealed by a suitable seal 73 (e.g., an O-ring, lip seal or the like). Reservoir 62B may have any of a wide variety of shapes and configurations. The interior of reservoir 62B is in fluid communication with a test chamber 12. Reservoir 62B may, for example, be mounted directly to a test chamber 12, beside the test chamber 12, etc.

The pressure of fluid 15 in reservoir 62B will depend on the mass of piston 70 as well as the cross-sectional area of piston 70. The mass and dimensions of piston 70 may be calculated with respect to the desired pressure. In one embodiment, the testing of prosthetic heart valves, a hydrostatic pressure of 70 mmHg is sufficient. A piston 70 made of tungsten alloy having a height of 5.5 cm and a uniform cross section would produce a hydrostatic pressure of 70 mmHg.

Pressure reference system 60B may be prepared for use by filling reservoir 62B with fluid 15 starting with piston 70 fully lowered. As fluid 15 is added into reservoir 62B the pressure in reservoir 62B will increase until piston 70 begins to rise. Additional fluid 15 may be added to reservoir 62B until piston 70 is lifted further to store additional fluid 15 in reservoir 62B.

Pressure reference system 60B may be designed to allow piston 70 to move through strokes of various lengths depending on the volume of fluid that it is desired to store in reservoir 62B. For example, reservoir 62B may be designed to hold sufficient fluid 15 to make up for expected fluid loss from a test chamber 12 in a specified period of operation. For example, 90 ml fluid 15 would suffice to replenish fluid 15 in a test chamber 12 for 30 days at a depletion rate of 3 ml/day. A 90 ml volume could be provided by a pressure reference system 60B having a piston having a diameter of 5 cm and a stroke length of about 4.6 cm.

Piston 70 may be constructed in any of various ways and of any of a wide range of materials. For example, piston 70 may be made of:
a dense material such as tungsten (19.25 g/cm2)—this construction allows a desired pressure to be generated with a smaller piston 70;
a less dense material such as stainless steel (7.9 g/cm3)—this requires a larger piston 70 for the same pressure but may be more cost effective than a denser material; and/or
a combination of different materials such as a denser material coated or encased in a less dense material such as a suitable plastic.

In some embodiments piston 70 carries a separate weight 71 which may have cross sectional dimensions different from piston 70, as shown in the example embodiment in FIG. 20. In some such embodiments, piston 70 is made of a low density material such as a suitable plastic (e.g., acetal, Teflon™, UHWM plastic or the like). The mass of weight 71 may be selected to achieve a desired pressure in reservoir 62B.

FIG. 21 schematically illustrates a pressure reference system 60C that is similar to pressure reference system 60B except that piston 70 is sealed by a rolling diaphragm 72. Pressure reference system 60C otherwise operates in the same manner as pressure reference system 60B.

Some advantages of the use of a rolling diaphragm 72 over a dynamic seal are that piston 70 is isolated from contact with fluid 15 (which may be corrosive to some materials) and piston 70 may be made to looser tolerances where a seal is provided by a rolling diaphragm. A rolling diaphragm configured as shown in FIG. 21 can also limit the maximum amount of fluid stored in reservoir 62B.

Apparatus as described herein may be applied, for example, to testing aortic or mitral prosthetic heart valves.

Where a component (e.g. a software module, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Motion controllers and computers in various embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, and the like. For example, one or more data processors in a control circuit for a valve testing apparatus may implement methods as described herein by executing software instructions in a program memory accessible to the processors.

Certain aspects of the invention may also be provided in the form of a program product. The program product may comprise any non-transitory medium which carries a set of computer-readable instructions which, when executed by a data processor, cause the data processor to coordinate execution of a method of the invention (e.g., by controlling apparatus as described herein and/or processing measurements as described herein. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

### Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
“about” when applied to a numerical value means the numerical value±20%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the total number of referenced elements.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, as well as all other numerical values that are near to or approximately equal to that numerical value. A particular numerical value is included in “about” a specifically recited numerical value where the particular numerical value provides the substantial equivalent of the specifically recited numerical value in the context in which the specifically recited numerical value is presented.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs or sentences. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. Apparatus for testing prosthetic valves comprising:
a test chamber divided into an inflow chamber and an outflow chamber, the test chamber having a fluid connection path between the inflow chamber and the outflow chamber;
a mount for supporting a prosthetic valve under test in the fluid connection path with the prosthetic valve oriented to open to pass flow on the fluid connection path from the inflow chamber toward the outflow chamber and to close to restrict flow on the fluid connection path toward the inflow chamber from the outflow chamber;
a fluid driving device operable to vary a pressure differential of a fluid between the inflow chamber and the outflow chamber in repeating cycles which include an open phase in which the valve under test is open and a closed phase in which the valve under test is closed; and
wherein the fluid driving device is configured to drive the fluid into the outflow chamber to increase a pressure in the outflow chamber in the closed phase and to drive the fluid out of the outflow chamber to decrease the pressure in the outflow chamber in the open phase.

2.-3. (canceled)

4. The apparatus according to claim 1 wherein: the fluid driving device varies the pressure differential in repeating cycles at least 200 times per minute.

5. The apparatus according to claim 1 comprising a source of fluid at the inflow chamber, the source of fluid configured to maintain a mean fluid pressure in the inflow chamber.

6. The apparatus according to claim 5 wherein the source of fluid comprises a reservoir and a conduit fluidly connecting the reservoir to the inflow chamber.

7. (canceled)

8. The apparatus according to claim 6 wherein an end point of the conduit in the inflow chamber is not more than 40 mm from the mount.

9. The apparatus according to claim 6 wherein the reservoir is configured to contain fluid with a surface elevation of the fluid at least 60 mm higher than the mount.

10. The apparatus according to claim 9 wherein the surface elevation does not exceed 500 mm above the mount.

11. The apparatus according to claim 9 wherein the surface elevation is in the range of 60 mm to 1.5 m above the mount.

12.-16. (canceled)

17. The apparatus according to claim 1 wherein the test chamber has a first end that defines a wall of the inflow chamber, a second end that defines a wall of the outflow chamber and sides extending between the first and second ends and wherein the fluid driving device is fluidly coupled to the inflow chamber by an inflow channel that extends into the inflow chamber through one of the sides.

18. The apparatus according to claim 17 wherein the fluid driving device cyclically drives the fluid in or out of the inflow chamber through the inflow channel.

19. The apparatus according to claim 17 wherein the fluid driving device is fluidly coupled to the outflow chamber by an outflow channel that extends into the outflow chamber through one of the sides.

20. (canceled)

21. The apparatus according to claim 1 wherein the fluid driving device comprises a reciprocating piston and the reciprocating piston is operative to push fluid into the outflow chamber to increase the pressure in the outflow chamber and pull fluid out of the outflow chamber to decrease the pressure in the outflow chamber.

22. (canceled)

23. The apparatus according to claim 1 wherein the fluid driving device comprises a reciprocating bellows and the reciprocating bellows is operative to push fluid into the outflow chamber to increase the pressure in the outflow chamber and pull fluid out of the outflow chamber to decrease the pressure in the outflow chamber.

24.-103. (canceled)

104. The apparatus according to claim 1 comprising:
a bypass valve comprising a variable aperture arranged to allow fluid to flow between the outflow chamber and the inflow chamber;
a first sensor operative to measure a pressure of the fluid in the inflow chamber;
a second sensor operative to measure a pressure of the fluid in the outflow chamber;
a control system configured to:
store an upper control limit and a lower control limit,
determine a value for a target variable based at least on output signals from the first and second sensors, and
in response to a relationship of the target variable value to the upper and lower control limits, control an actuator to incrementally open or close the bypass valve so as to cause the target variable value to be varied to increase the target variable value if the target variable value is below the lower control limit or to decrease the target variable value if the target variable value is above the upper control limit.

105. The apparatus according to claim 104 wherein the target variable is a peak differential pressure across the prosthetic valve.

106. The apparatus according to claim 104 wherein the target variable is a high pressure time for the differential pressure across the prosthetic valve.

107. The apparatus according to claim 104 wherein the target variable is a cycle passing rate for the prosthetic valve.

108. The apparatus according to claim 107 wherein the cycle passing rate is calculated based on a plurality of past test cycles.

109. The apparatus according to claim 104 wherein the target variable value is controlled to be at a midpoint between the upper control limit and the lower control limit.

110. The apparatus according to claim 104 wherein the target variable value is controlled to be at or slightly above the lower control limit.

111. The apparatus according to claim 104 wherein the target variable value is controlled to be between the lower control limit and the mean of the upper and lower control limits.

112. The apparatus according to claim 104 wherein the actuator comprises a servomotor or a stepper motor or a linear actuator that is mechanically coupled to operate the bypass valve.

113. The apparatus according to claim 104 wherein the bypass valve is a plug valve.

114.-115. (canceled)

116. The apparatus according to claim 113 wherein a plug of the plug valve has first and second axial end portions that are smaller in diameter than a portion of the plug between the first and second axial end portions.

117. The apparatus according to claim 116 wherein the first and second axial end portions are tapered.

118.-122. (canceled)

123. The apparatus according to claim 104 comprising an encoder connected to measure the degree of rotation of the plug valve and transmit a signal indicating a degree of rotation to the control system.

124. The apparatus according to claim 104 comprising a user interface operative to receive a user input specifying a value for one or both of the upper control limit and the lower control limit.

125. The apparatus according to claim 104
wherein the target variable comprises a rolling cycle passing rate and the control system is configured to:
receive sensor data indicating a fluid pressure in the inflow chamber and a fluid pressure in the outflow chamber;
process the sensor data to assess whether or not each cycle of the fluid driving device passed requirements for peak differential pressure and high pressure time;
compute the rolling cycle passing rate; and
control the bypass valve based on the rolling cycle passing rate to maintain the rolling cycle passing rate between the lower control limit and an upper control limit.

126. The apparatus according to claim 125 wherein the upper control limit is less than 100%.

127. The apparatus according to claim 126 wherein the upper control limit is greater than 99%.

128. The apparatus according to claim 128 wherein the lower control limit is equal to or greater than 95%.

129. The apparatus according to claim 125 wherein the control system is configured to control the bypass valve to maintain the rolling cycle passing rate incrementally above the lower control limit.

130. The apparatus according to claim 125 wherein the rolling cycle passing rate is determined for a window containing passing information for at least the most recent 900 cycles.

131.-148. (canceled)
